(19) United States
(12) Patent Application Publication
(10) Pub. No.: US 2008/0058595 A1
(43) Pub. Date: Mar. 6, 2008

(54) Medical device introduction systems and methods

(76) Inventors: Phillip Jack Snoke, Winston-Salem, NC (US); Joe B. Massey, Atlanta, GA (US); Marcus E. Finch, Irmo, SC (US); John A. McMillan, Atlanta, GA (US); Mark Curran Martel, Belews Creek, NC (US); Kenneth Todd Cassidy, Mocksville, NC (US); Philip Allred, High Point, NC (US); Jon S. Wilson, Lewisville, NC (US); Lawrence B. Rothstein, Dayton, OH (US)

Correspondence Address: KILPATRICK STOCKTON LLP, 1001 WEST FOURTH STREET, WINSTON-SALEM, NC, 27101, US

(21) Appl. No.: 11/811,957

(22) Filed: Jun. 13, 2007

Related U.S. Application Data
(60) Provisional application No. 60/813,807, filed on Jun. 14, 2006.
(60) Provisional application No. 60/878,230, filed on Jan. 3, 2007.

(51) Int. Cl. A61B 1/00

(52) U.S. Cl. 600/114

(57) ABSTRACT

A medical device introduction system and method can include a medical introducer, a separate imaging device, and/or a separate working channel device, each of which may be movable independent of the other. The medical introducer can include a handle and an elongate introducer tube extending from the handle and having a plurality of lumens, and may be inserted into an interior body region of a patient. The separate imaging device may be inserted through the handle and positioned in one of lumens. The separate working channel device can include an elongate working channel tube and a position controller. The working channel tube can include at least one lumen defining a working channel. The position controller can be configured to control positioning of the working channel tube. The working channel device may be removably connected to the handle and positioned in another lumen.

### CROSS-REFERENCE TO RELATED APPLICATIONS

This application claims benefit of U.S. Provisional Patent App. No. 60/813,807, filed Jun. 14, 2006, and U.S. Provisional Patent App. No. 60/878,230 filed Jan. 3, 2007, each of which is incorporated by reference herein in its entirety.

### FIELD OF THE INVENTION

The present invention relates to medical device introduction systems and methods. Embodiments of the present invention may be useful for separately introducing and independently controlling multiple cooperating medical devices in interior body regions.

### BACKGROUND OF THE INVENTION

In recent years, medical procedures have advanced to stages where less and less invasive, or minimally invasive, surgeries, diagnostic procedures, exploratory procedures, or other medical procedures have been desired and demanded by patients, physicians, and payers. To accomplish these desires and demands, various medical devices and instrumentation have been developed, such as cannulas or micro-cannulas, various catheter devices, micro-surgical instrumentation and implants, medical introducers, imaging devices such as fiberoptic scopes, and other related endoscopic devices.

In situations in which minimally invasive procedures are used, space within an interior body region, for example, an organ, opening, cavity, passageway, or vessel, can become more and more constrained. As a result, operating within small spaces with a plurality of medical devices, such as scopes, dilating and cutting instruments, fluids, catheters, implants, and the like, can become difficult to manage. When performing a procedure with a plurality of medical devices, positioning, controlling, manipulating, and handling the various medical devices during the procedure can limit a physician's ability to perform as well as capable. That is, the design and construction of a medical device can limit a physician's ability to view a target site, maneuver within a space, transition between procedures, and/or perform additional procedures. Managing the use of multiple devices in a procedure can pose even greater difficulty to a single physician who desires to perform a procedure, often without assistance or with limited assistance, in an office or outpatient setting so as to avoid the time and expense of hospital utilization for such procedures.

Conventional medical devices having optical capabilities, such as conventional endoscopes, can have other disadvantages. The optical capabilities can be limited due to various factors, including, for example, the anatomical structures about which the scopes are maneuvered, and the movement and/or control together of both the imaging device and a delivery device and the resulting loss in orientation in an interior body region. For example, optical capabilities with conventional endoscopes typically used in hysteroscopy procedures are often limited in such ways, making it difficult for the physician to know whether what is being viewed is up or down. Such conventional endoscopes and associated delivery devices are often complex and require extended learning to operate effectively. In addition, many conventional endoscopes and delivery devices are reusable and can be very expensive to purchase and to re-sterilize after each use. As a result, physicians often elect not to perform diagnostic and/or therapeutic procedures in a medical office or outpatient setting that could otherwise be performed to the patient's advantage in those settings.

During use in medical procedures, introducer instruments, sheaths, endoscopes, and working catheters and cannula can be exposed to various bacteria, viruses, and other microorganisms, and to potentially disease carrying media. These microorganisms can be trapped in such devices, particularly in lumens, and transferred to subsequent patients or users. Sterilization methods can be employed on such devices that are reusable in an attempt to disinfect and eliminate microorganisms for subsequent use of the devices. However, some surgical devices contain very small and/or narrow working channels or lumens for performing intricate medical procedures. These small and/or narrow working channels can be difficult to clean and sterilize. If not effectively eliminated, these materials may be transferred to, and potentially cause harmful infections to, other patients or medical personnel through subsequent use of the devices.

In addition to the problems of potential disease transmission and lack of disposability, conventional reusable medical introducer, endoscopes, and the like are subjected to repeated use over prolonged periods. The precision of manipulation and movement in endoscopes and steerable medical devices is often essential for conducting complicated diagnostic and therapeutic medical procedures generally performed with such devices. Some reusable devices containing steering mechanisms often require precision calibration. Further, these devices are regularly subjected to sterilization with heat or chemicals. To accomplish these objectives, conventional reusable devices are often made of stainless steel or other durable materials that are costly. In addition, despite being designed for repeated use, such conventional intricate reusable devices, in particular, such devices that incorporate visualization components, often require regular replacement, further adding to the cost of such devices.

### SUMMARY

Some embodiments of an medical device introduction system and method of the present invention can include a medical introducer, a separate imaging device, and/or a separate working channel device. The medical introducer can include a handle and an elongate introducer tube extending from the handle. The introducer tube can include a plurality of lumens extending longitudinally therein. The medical introducer may be inserted into an interior body region of a patient. The separate imaging device may be inserted through the handle and positioned in a predetermined one of the plurality of lumens. The imaging device can have an interface with the handle such that each of the imaging device and the medical introducer is movable independent of the other. The separate working channel device can include an elongate working channel tube and a position controller. The working channel tube can include at least one lumen extending the length thereof defining a working channel. The position controller can be configured to control positioning of the working channel device or tube. The working channel device may be removably connectable to the handle and positioned in another predetermined one of the plurality of lumens. In some embodiments of the present invention, each of the medical introducer, the imaging device, and the working channel device can be movable independent of the other.

In certain embodiments, the medical introducer handle can comprise an oval-shaped ring of material having an open interior. The handle can have a proximal end configured to receive at least one fluid tube and the imaging device therethrough. The handle can further include a distal end adapted to connect to the introducer tube. In certain embodiments, the plurality of lumens in the introducer tube can include a scope lumen, at least one working lumen, and at least one fluid lumen separate from the scope lumen and the working lumen(s). In an illustrative embodiment, the medical introducer can further include a fluid inflow tube routed through the proximal end of the handle and in fluid communication with a fluid lumen, and a fluid outflow tube routed through the proximal end of the handle and in fluid communication with another fluid lumen. In some embodiments, the medical introducer can include a modular manifold integrally formed on the proximal end of the introducer tube and have a corresponding plurality of lumens aligned with the plurality of lumens in the introducer tube. The manifold can be removably connected to the introducer handle such that the manifold and introducer tube are interchangeable in the handle with other manifolds and introducer tubes.

In some embodiments, the medical introducer and/or the working channel device can be disposable. In some embodiments, at least a portion of the medical introducer and/or at least a portion of the working channel device can be translucent such that passage of materials therethrough is viewable.

In some embodiments, one or more of the introducer tube, the working channel tube, and the endoscopic cannula can include a proximal portion having a first durometer and a distal portion having a second durometer. The second durometer is lower than the first durometer so as to allow deflection of the distal portion of the respective tube or cannula for controllable access to a target area in the interior body region. In some embodiments further durometer(s) may be utilized to enhance steering.

In certain embodiments, the working channel device can be a steerable working channel device. In such an embodiment, the working channel tube can comprise a flexible distal portion adapted for steering to selected positions. The position controller can be operably connected to the working channel tube distal portion and slidable within the introducer handle for moving the working channel tube distal portion in distal and proximal directions. In addition, the position controller can be actuated to steer the flexible distal portion of the working channel tube in predetermined directions and amounts.

In some embodiments, the imaging device can include an endoscopic cannula, a light delivery mechanism, and a imaging system. The light delivery system can comprise light emitting diodes and/or light delivery fibers. The imaging system can be an optical scope, an ultrasound instrument, or a camera.

In some embodiments, a medical introducer and/or a working channel device are configured to provide a user an ability to establish a predetermined route of delivery. The medical introducer and/or working channel device may be further configured to provide alternative routes of delivery. The delivery route and/or routes may be established, for example visually, using components of the medical introducer and/or working channel device, for example through locking, braking or fixing of components.

The present invention can include embodiments of a method. For example, a medical introducer comprising a handle and an elongate introducer tube extending therefrom and having a plurality of lumens extending longitudinally therein can be inserted into an interior body region of a patient. A separate imaging device can be inserted through the handle and in a predetermined one of the plurality of lumens. The imaging device can be positioned in a selected position within the interior body region. Then, an image can be produced from within the interior body region. A separate working channel device and position controller can be removably connected to the medical introducer. The working channel device can include an elongate working channel tube having at least one lumen extending the length thereof defining a working channel. The position controller for controlling the position of the working channel tube can be positioned in the working channel in another predetermined one of the plurality of lumens. In such embodiments, one of the group of the medical introducer, the imaging device, and the working channel device may be moved independently of the others of the group.

In some embodiments of a method, the medical introducer handle can comprise an oval-shaped ring of material having an open interior. The method can further include connecting a distal end of the handle to the introducer tube. In some embodiments of a method, the medical introducer can include a modular manifold integrally formed on a proximal end of the introducer tube and have a corresponding plurality of lumens aligned with the plurality of lumens in the introducer tube. In such an embodiment, the manifold can be removably connected to the introducer handle. The manifold and introducer tube may be interchanged in the handle with other manifolds and introducer tubes.

Certain embodiments of a method of the present invention include performing a medical procedure in an interior body region through the working channel device. For example, the medical procedure can be a gynecological procedure, a spinal procedure, or other procedure.

### BRIEF DESCRIPTION OF THE DRAWINGS

FIG. 1 is a perspective view of a medical device introduction system in an embodiment of the present invention.

FIG. 2 is a perspective view of the medical introducer shown in FIG. 1, showing a plug seal in the working channel in an embodiment of the present invention.

FIG. 3 is a view of the medical introducer shown in FIG. 1, showing a plug adapter with seal in the working channel in an embodiment of the present invention.

FIG. 4 is a close-up, perspective view of the manifold of the medical introducer shown in FIG. 1, in an embodiment of the present invention.

FIG. 5 is a cross-sectional view taken along the lines 5-5 of the lumens in the manifold shown in FIG. 4, in an embodiment of the present invention.

FIG. 6 is a close-up, perspective view of the steerable working channel device position controller shown in FIG. 1, in an embodiment of the present invention.

FIG. 7 is a cross-sectional view taken along the lines 7-7 in FIG. 6 of the internal components of the steerable working channel device position controller in an embodiment of the present invention.

FIG. 8 is a close-up perspective view of the steerable working channel device proximal port shown in FIGS. 1 and 6 in an embodiment of the present invention.

FIGS. 9A-9E are top views of the medical introducer and steerable working channel device shown in FIG. 1, in an embodiment of the present invention. FIG. 9A shows the position controller in distal position and the distal end portion deflected to the left. FIG. 9B shows the position controller in medial position and the distal end portion deflected to the left.

FIG. 9C shows the position controller in proximal position and the distal end portion fully retracted. FIG. 9D shows the position controller in medial position and the distal end portion deflected to the right. FIG. 9E shows the position controller in distal position and the distal end portion deflected to the right.

FIG. 10 is a view of the medical introducer and steerable working channel device shown in FIG. 1, illustrating positioning of the steerable working channel tube in a uterine cavity in an embodiment of the present invention.

FIG. 11 is a close-up view of the endoscope and camera shown in FIG. 1, in an embodiment of the present invention.

FIG. 12 is a cross-sectional view of a steerable working channel showing steering wire lumens and areas of the working channel tube having different relative durometers in an embodiment of the present invention.

FIG. 13 is a view of a medical introducer tube having a lift wire lumen in an embodiment of the present invention.

FIG. 14 is a view of a medical introducer tube showing a lumen configuration having a large scope lumen and three smaller lumens for delivering a medical device and for fluids in an embodiment of the present invention.

FIG. 15 is a side view of a medical device introduction system having an accessory device support attached thereto, the accessory device support supporting an implant delivery device, in an embodiment of the present invention.

FIG. 16 is a view of a continuous flow examination sheath useful in an embodiment of the present invention, showing both assembled and unassembled views.

FIG. 17 is a view of a single flow examination sheath useful in an embodiment of the present invention, showing both assembled and unassembled views.

FIG. 18 is a view of a preformed delivery tube useful in an embodiment of the present invention, showing both assembled and unassembled views.

### DETAILED DESCRIPTION

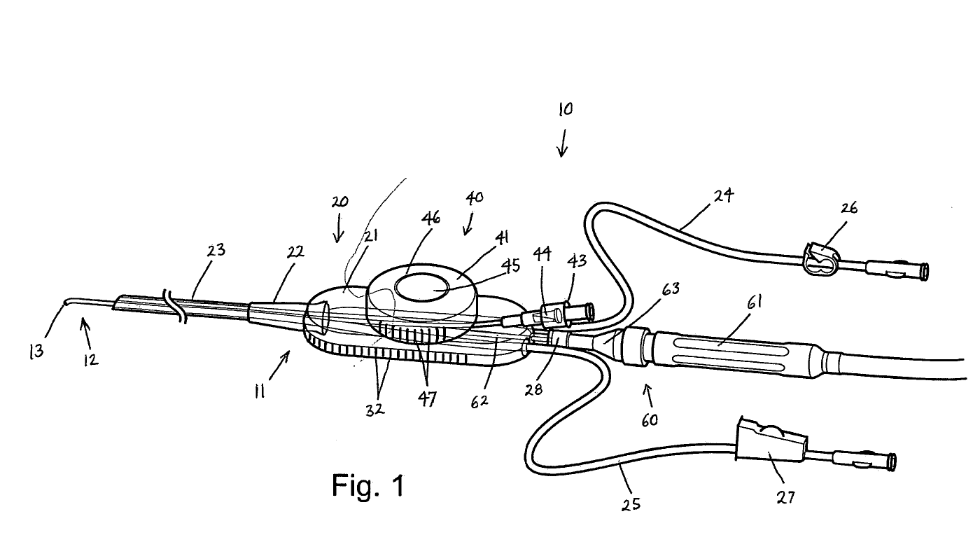

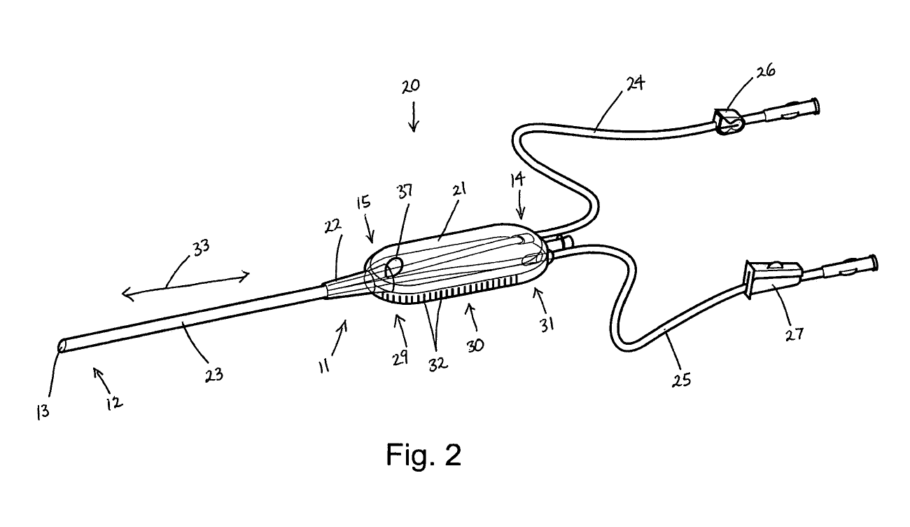

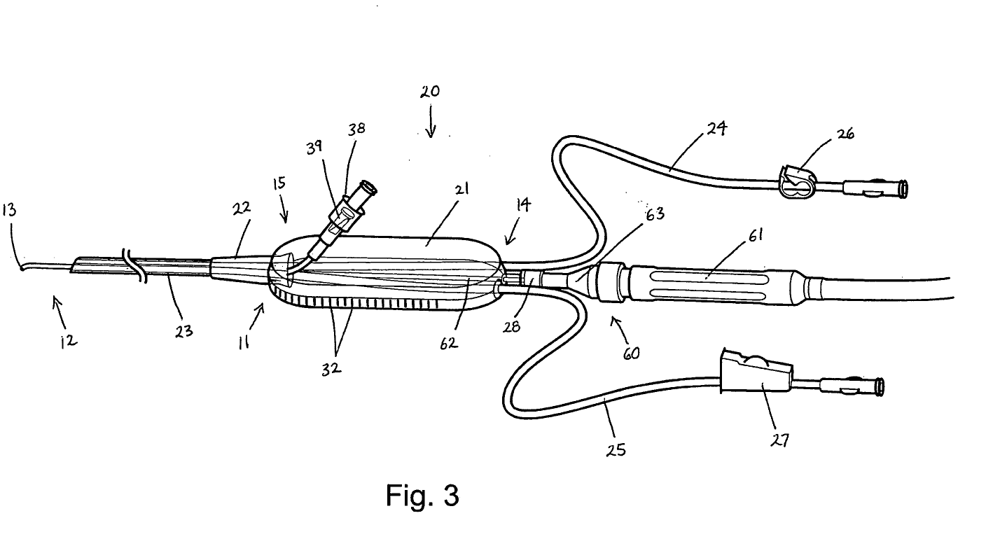

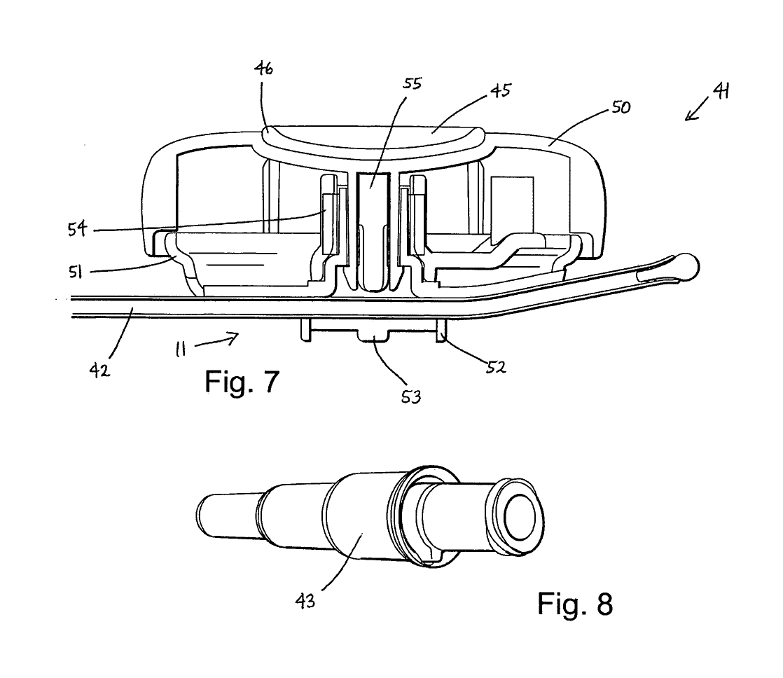

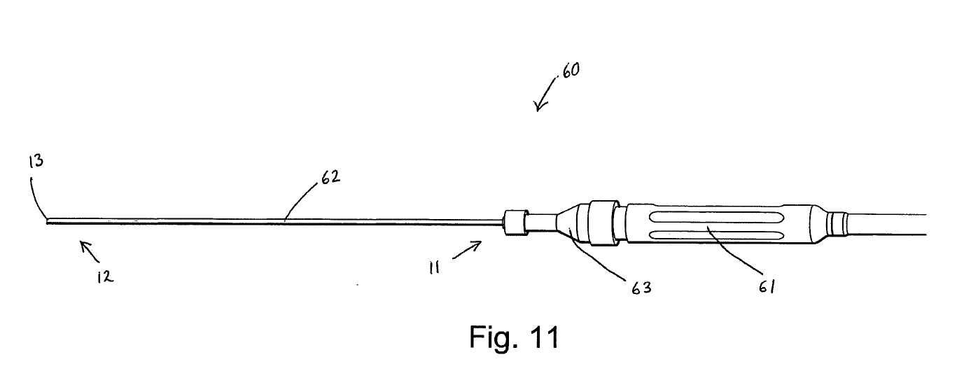

Some embodiments of the present invention can provide a medical device introduction system and/or method. FIGS. 1-18 show various aspects of such embodiments. For example, an illustrative embodiment of a medical device introduction system and/or method can include a medical introducer, a separate imaging device, and/or a separate working channel device. In such an embodiment, each of the medical introducer, the imaging device, and the working channel device can be movable independent of the other.

Minimally invasive surgical procedures have been developed that can be used in many diagnostic and/or therapeutic medical procedures. Such minimally invasive procedures can reduce pain, post-operative recovery time, and the destruction of healthy tissue. In minimally invasive surgery, the site of pathology can be accessed through portals rather than through a significant incision, thus preserving the integrity of intervening tissues. These minimally invasive techniques also often require only local anesthesia.

Some embodiments of the present invention can provide systems, devices, kits, and methods useful for easily and effectively accomplishing minimally invasive gynecological procedures, for example, a hysteroscopy. Such systems, devices, kits, and methods may be adapted for use in many interior body regions, wherever introduction of medical devices may be required for a therapeutic or diagnostic purpose.

As used in this specification and the appended claims, “proximal” is defined as nearer to a point of reference such as an origin, a point of attachment, or the midline of the body. As used in this specification and the appended claims, “distal” is defined as farther from a point of reference, such as an origin, a point of attachment, or the midline of the body. Thus, the words “proximal” and “distal” refer to, for example, direction nearer to and farther from, respectively, an operator (for example, surgeon, physician, nurse, technician, etc.) who inserts a medical device into a patient, with the distal end, or tip, of the device inserted inside the patient's body. For example, the end of a medical device inserted inside the patient's body is the distal end of the medical device, while the end of the medical device outside the patient's body is the proximal end of the medical device.

As used in this specification and the appended claims, the singular forms “a,” “an,” and “the” include plural referents unless the context clearly dictates otherwise. Thus, for example, the term “a lumen” is intended to mean a single lumen or a combination of lumens. For the purposes of this specification and the appended claims, unless otherwise indicated, all numbers expressing quantities, conditions, and so forth used in the specification are to be understood as being modified in all instances by the term “about.” Accordingly, unless indicated to the contrary, the numerical parameters set forth in this specification are approximations that can vary depending upon the desired properties sought to be obtained by embodiments of the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of embodiments of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a stated range of “1 to 10” should be considered to include any and all sub-ranges between (and inclusive of) the minimum value of 1 and the maximum value of 10. That is, a stated range of “1 to 10” should be considered to include, for example, all sub-ranges beginning with a minimum value of 1 or more, such as 1 to 6.5, and ending with a maximum value of 10 or less, such as 5.5 to 10. Additionally, any reference referred to as being “incorporated herein” is to be understood as being incorporated in its entirety.

As used in this specification and the appended claims, an “interior body region” can be a body cavity, a body space or potential space, a vein, an artery, a vessel, a duct, a pathway, an organ, or any interior site in a patient's body accessible with a medical introducer.

As used in this specification and the appended claims, an endoscope is defined as an instrument for examining an interior body region. Endoscopes are generally tools used to view within a portion of the anatomy through an open end of a tube. Flexible endoscopes may be utilized in certain deformable anatomical structures, for example, arteries, ureters, and the common bile duct. Endoscopes can be used to look directly through an objective lens or in conjunction with video cameras attached remotely to the scope for viewing a portion of the human body. Rod lens systems may also be used with some endoscopes to view images. In other endoscopes, the image may be gathered at the distal end by a lens and transferred to a proximal objective lens using fiber optic bundles.

Some embodiments of a medical device introduction system 10 and method of the present invention can include a medical introducer 20, a separate imaging device 60, and/or a separate working channel device 40. The medical introducer 20 can include a proximal end 11, a distal end 12, a handle 21, and an elongate introducer tube 23 extending from the handle 21. The introducer tube 23 can include a plurality of lumens extending longitudinally therein. The medical introducer 20 may be inserted into an interior body region of a patient. The separate imaging device 60 may be inserted through the handle 21 and positioned in a predetermined one of the plurality of lumens. The imaging device 60 can have an interface with the handle 21 such that each of the imaging device 60 and the medical introducer 20 is movable independent of the other. The separate working channel device 40 can include an elongate working channel tube 42 and a position controller 41. The working channel tube 42 can include at least one lumen extending the length thereof defining a working channel. The position controller 41 can be configured to control positioning of the working channel tube 42. The working channel device 40 may be removably connectable to the handle 21 and positioned in another predetermined one of the plurality of lumens. In some embodiments of the present invention, each of the medical introducer 20, the imaging device 60, and the working channel device 40 can be movable independent of the other.

In such an embodiment, the imaging device 60 can be placed into a desired position for viewing a procedure. The imaging device 60, such as the camera 61, can be held in a steady, or fixed, position, while the distal portion 12 of the steerable working channel 40 can be positioned, or re-positioned, (extended, retracted, or deflected) independent of the imaging device 60. In this manner, the starting reference point, such as the “horizon” and/or depth of the steerable working channel 40 in the interior body region can be held constant by the imaging device 60. As a result, the true movement of the steerable working channel relative to a certain starting point can be gauged. Alternatively, the steerable working channel device 40 can be held in a fixed position so as to maintain a fixed orientation, or reference point, of the working channel tube portion 42 of the working channel in the interior body region. While the steerable working channel device 40 is held in a constant position, the position of imaging device 60 can be adjusted independent of the steerable working channel device 40. In this manner, the true movement of the imaging device 60 relative to a certain starting point can be gauged.

In addition, while holding the imaging device 60 in a fixed position, the medical introducer 20 can be independently rotated about its longitudinal axis 33 if desired. Rotation of the medical introducer 20 may be desired for purposes such as adjusting the starting position of the steerable working channel tube 42 prior to extending or deflecting the distal tip of the working channel tube 42, or reorienting fluid outflow at a target area in the interior body region. In this manner, the true movement of the medical introducer 20 relative to a certain starting point can be gauged. Likewise, if desired, the medical introducer 20 and the attached steerable working channel device 40 can be held in a constant position so as to maintain a fixed orientation, or reference point, of the working channel tube 42 and the working channel in the interior body region. While the medical introducer 20 and the attached steerable working channel device 40 can be held in a constant position, the position of imaging device 60 can be adjusted. In this manner, the true movement of the imaging device 60 relative to a certain starting point can be gauged.

This combination of separate and cooperating components of embodiments of the present invention allows for more precise control of instrument positioning and delivery of materials, such as fluids, medications, and implants, in an interior body region. Independent position control and movement of the imaging device 60 relative to the medical introducer 20 and to the steerable working channel device 40 allows optimal visualization of a target operative site within an interior body region.

An embodiment of the medical device introduction system 10 of the present invention can include a medical introducer 20. As used herein, a “medical introducer” is defined as an instrument used to introduce a medical device, for example, a tube, stent, catheter, and/or surgical instrument, into an interior body region of a human or animal.

In some embodiments of the present invention, the medical introducer device 20 can include the handle 21 comprising an oval-shaped ring of material having an open interior, a proximal end, and a distal end. The introducer 20 can further include an elongate introducer tube 23 extending from the distal end 15 of the handle 21 and having a plurality of lumens extending longitudinally therein. The proximal end 14 of the handle 21 can be configured to receive at least one fluid tube 24, 25 and the imaging device 60 through the handle 21. The distal end 15 of the handle 21 can be adapted to connect to the introducer tube 23, as described herein. Such a medical introducer 20 can be inserted into an interior body region of a patient.

The plurality of lumens in a medical introducer tube 23 can include a scope lumen 34, at least one working lumen 35, and at least one fluid lumen 36 separate from the scope lumen 34 and the working lumen(s) 35. The medical introducer 20 can further include a fluid inflow tube 24 routed through the proximal end of the handle 21 and in fluid communication with one of the at least one fluid lumen 36. The medical introducer 20 can further include the fluid outflow tube 25 routed through the proximal end of the handle 21 and in fluid communication with another one of the fluid lumen(s) 36. In certain embodiments, the diameter of the working lumen 35 can be larger than the diameter of the other lumens 34, 36.

The medical introducer 20 can be utilized to perform diagnostic procedures, for example, by using the dedicated fluid-in and fluid-out lumens 36 and tubes 24, 25, respectively, to irrigate an interior body region and retrieve a sampling of washings from the targeted region for diagnostic tests. Alternatively, or in addition, the medical introducer 20 can be utilized to perform therapeutic procedures, for example, by using the dedicated working lumen 35 to introduce a device for placing a medication and/or an implant into an interior body region.

The fluid-in tube 24 can include a pinch clamp 26 for on-off regulation of fluid delivery into the interior body region. The fluid-out tube 25 can include a roller clamp 27 for graduated regulation of fluid flow out of the interior body region. In other embodiments, regulation of fluid flow on both the fluid-in tube 24 and the fluid-out tube 25 can be managed by different regulation mechanisms, for example an electronic fluid pump for fluid delivery or a suction device for fluid removal. Separate dedicated fluid lumens 36 and tubes 24, 25 in embodiments of the present invention can allow better fluid flow, for example, more continuous fluid flow, than conventional medical device introducers that often deliver fluid to an interior body region through a working lumen 35 in which a medical device may be placed simultaneously.

The medical introducer 20 can include a modular manifold 22 integrally formed on the proximal end of the introducer tube 23 and have a corresponding plurality of lumens aligned with the plurality of lumens in the introducer tube 23. The manifold 22 may be removably connected to the handle 21 such that the manifold 22 and introducer tube 23 are interchangeable in the handle 21 with other manifolds 22 and introducer tubes 23.

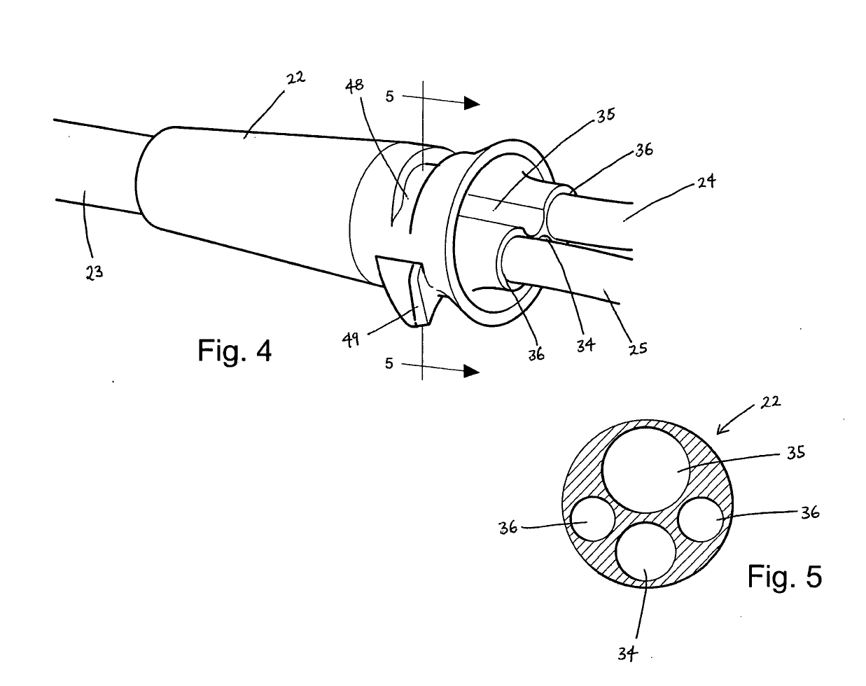

As shown in FIG. 4, the handle 21 can be connected to the manifold 22 by snapping a groove, or cut-out in the distal end 15 of the handle 21 about a correspondingly shaped handle receiving groove 48 in the manifold 22. The manifold 22 can include a handle support 49 extending downwardly from the bottom of the manifold below the handle receiving groove 48. When the handle 21 is removably snap fit about the handle receiving groove 48 in the manifold 22, the distal end 15 of the handle 21 can abut the handle support 49 to provide further support of the positioning of the handle 21 on the manifold 22.

In an embodiment in which a separate imaging device 60 is inserted through the handle 21 and positioned in a predetermined lumen (34) in the medical introducer 20, the imaging device 60 can have an interface with the handle 21 such that each of the imaging device 60 and the medical introducer 21 is movable independent of the other. In certain embodiments, the medical introducer device 20 can cooperate with a separate working channel device 40. The separate working channel device 40 can comprise an elongate working channel tube 42 having at least one lumen extending the length thereof defining a working channel and a position controller 41 for controlling the position of the working channel tube 42. The working channel device 40 can be removably connected to the handle 21 and positioned in a predetermined lumen (35) in the medical introducer 21 separate from the imaging device 60, such that each of the medical introducer 20, the imaging device 60, and the working channel device 40 is movable independent of the other.

In some embodiments, the medical introducer device 20 can be disposable. In some embodiments, at least a portion of the medical introducer device 20 can be translucent such that passage of materials therethrough can be viewed.

The introducer tube 23 can include a proximal 11 portion having a first durometer and a distal portion 12 having a second durometer. As used herein, durometer is defined as a degree of hardness; a harder material comprises a higher durometer than a softer material. The second durometer can be lower than the first durometer so as to allow deflection of the distal portion 12 for controllable access to a target area in the interior-body region. The introducer tube distal portion can include a distal tip 13 having a first diameter smaller than a second diameter of the remainder of the introducer tube 23, such that the smaller first diameter is adapted to seal about a device extending beyond the distal tip 13. The introducer tube 23 can further include a fluid lumen 36 comprising a wall having a third durometer that is higher than the second durometer of the distal portion 12 so as to prevent collapsing of the fluid lumen 36 when the distal portion 12 of the introducer tube 20 is deflected.

In some embodiments, the introducer handle 21 can have a size adapted to be readily held in a hand of a user. In some embodiments, the introducer handle 21 can further include a plurality of raised grips 32 on an outside surface of the handle 21 to assist in manipulating the handle 21.

Embodiments of the medical introducer 20 can have varied numbers, sizes, and configurations of lumens 34, 35, 36 in the introducer 20. Embodiments of the medical introducer 20 can have various lengths, depending on the particular interior body region it is designed to access and on the particular medical procedure for which it is designed. For example, in some embodiments, the medical introducer 20 can include a 7 French size dedicated working lumen 35 so as to support passage of larger devices than conventional multiple lumen delivery devices having the same outside diameter. This advantage is provided by having a smaller dedicated scope lumen 34 and extruding the manifold 22 and introducer tube 23 with smaller wall thicknesses.

In certain embodiments, the introducer handle 21 can include a scope connector 28 located on the proximal end 14 of the handle 21. The scope connector 28 can be longitudinally aligned with the one of the plurality of lumens (34) in the introducer tube 23. The imaging device 60 can be securely connected to the scope connector 28, for example, with a luer lock fitting. When the imaging device 60 is securely connected to the scope connector 28, the imaging device 60—medical introducer 20 interface is adapted to allow the imaging device 60 to rotate independent of movement of the medical introducer 20.

The medical introducer 20 can be formed in a molding process by a plastic or polymeric material. The medical introducer 20 can be formed from materials and in such a manner so as to have most, or all, components be translucent, thereby enabling visualization and visually-guided passage of instruments and fluids through the introducer 20. Such visualization may also assist with establishing delivery routes as discussed herein. Further, such visualization may allow for the identification of a gaseous material (e.g. air) within a channel, and/or confirmation of the absence of such gaseous material within a channel.

The lumen 35 in the medical introducer 20 designed for inserting the steerable working channel device 40 can be sealed with a sealing mechanism. Such a seal 37 can be a duckbill seal or a one-way valve, including a luer fitting. The seals 37 can provide frictional or abutting contact with the inner surface of the working lumen 35 in the manifold 22. Such a seal 37 mechanism can allow medical devices and/or fluid, for example, gas or liquid, to pass through the seal mechanism 37 toward the distal end of the introducer tube 23, and can inhibit fluid from passing from the interior body region through the proximal end 11 of the introducer tube 23.

In certain embodiments, the medical introducer 20 can be inserted into an interior body region with a trocar system (not shown). A trocar can comprise a cannula that may have a sharp distal tip for creating a percutaneous path to the interior body region. Once the trocar is in a desired position in or adjacent the target interior body region, the medical introducer 20 can be inserted through the trocar to the target site. In such an application, a portion of the patient's body needs to be penetrated or opened where a body cavity does not provide a ready opening. Such a trocar system can be used, for example, for prostate surgery. In this manner, a trocar system, or other endoscopic device, can assist in providing a path through which the medical introducer 20 can enter the portion of the interior body region of a patient into which a medical procedure is desired to be performed.

The medical introducer 20 can be utilized to perform diagnostic procedures, for example, by using the dedicated fluid-in and fluid-out lumens 36 and tubes 24, 25 to irrigate an interior body region and retrieve a sampling of washings from the targeted region. Alternatively, or in addition, the medical introducer 20 can be utilized to perform therapeutic procedures, for example, by using the dedicated working lumen 35 to introduce a device for placing an implant into an interior body region.

In an alternative embodiment, the medical introducer 20 can further include an inflatable portion associated with the distal portion of the introducer tube 23. The inflatable portion can be utilized to distend or enlarge a cavity, space, or portion of an interior body region and/or block fluid passage from the interior body region when the introducer tube 23 is positioned therein.

In another aspect of the present invention, some embodiments can include a working channel device 40 that is steerable. The entire length of the working channel tube 42 can be flexible. Alternatively, a substantial portion of the working channel tube 42 can be generally rigid, or semi-rigid, and a distal portion 12 of the working channel tube 42 can be flexible. In such embodiments, as shown in FIGS. 1, 6, 7, and 9, the working channel tube 42 can include a flexible distal portion 12 adapted for steering to selected positions. In such embodiments, the position controller 41 can be operably connected to the working channel tube distal portion 12 and slidable within the introducer handle 21 for moving the working channel tube distal portion 12 in distal and proximal directions. In addition, the position controller 41 can be actuatable to steer the flexible distal portion 12 of the working channel tube 42 in predetermined directions and amounts. For example, the predetermined direction of steering can be in a plane generally parallel to an upper surface of the position controller 41.

In embodiments of a steerable working channel device 40, the device 40 can include, for example, at least two steering wires (not shown). Each steering wire has a distal end connected to the distal tip 13 of the working channel tube 42. Each steering wire can extend through the working channel tube 42, and have a proximal end operably connected to the position controller 41. In this way, the position controller 41 can be actuated to manipulate the distal portions 12 of the working channel tube 42.

In certain embodiments of the steerable working channel tube 42, the position controller 41 can further include a circular, lower housing 51 having an upwardly extending hollow hub 54 and a cooperating circular, upper housing 50 having a downwardly extending rotor 55 rotatingly seated inside the hollow hub 54. Each of the steering wires can be connected to an opposite side of the position controller rotor 55 such that rotation of the upper housing 50 causes rotation of the rotor 55 inside the hub 54, resulting in the distal end of the steering wire on one side of the rotor 55 to retract so as to deflect the distal tip 13 at an angle laterally away from the longitudinal axis 33 of the working channel tube 42.

As described herein, the introducer handle 21 can comprise an oval-shaped ring of material having an open interior. The open handle 21 can have a plurality of detents (not shown) on the inner surface of the handle 21 from the proximal position 31 to the medial position 30 to the distal position 29. The lower housing 51 of the position controller 41 can further include a downwardly extending bracket 52 adapted to friction fit in the inner surface of the handle 21 and a securing flange 53 extending outwardly from the bracket 52 adapted to friction fit about a bottom of the handle 21. Accordingly, the position controller 41 can be slidingly engageable with the detents (not shown) so as to secure the position of the working channel tube distal portion 12 and distal tip 13 along the longitudinal axis 33 of the working channel tube 42.

The position controller 41 can further include an automatic braking mechanism (not shown). For example, the braking mechanism can comprise a soft material on the outer surface of the upper housing rotor 55 and/or the outer surface of the lower housing hub 54 so as to provide sufficient friction to hold the upper housing 50 in position relative to the lower housing 51 when released by a user.

In some embodiments, the working channel tube 42 can further include a proximal 11 portion having a first durometer and a distal 12 portion having a second durometer. The second durometer can be lower than the first durometer so as to allow deflection of the distal portion 12 for improved access to a target area in the interior body region. The working channel tube distal portion 12 can further include the distal tip 13 having a first diameter smaller than a second diameter of the remainder of the working channel tube 42. The smaller first diameter can be adapted to seal about a device extending beyond the distal tip 13. Each or either of the proximal or distal portions may, in some embodiments, comprise a plurality of durometers to enhance steering.

It should be appreciated that other mechanisms for steering, for example, two finger deflection, may be utilized in some embodiments without departing from the present invention.

In certain embodiments, the working channel device 40 can further include at least one access port 38 having a seal 39. The sealed access port 38 can be connected to the proximal end 11 of the working channel tube 42 for controllable access to the steerable working channel.

The position controller 41 can have a size adapted to be readily held in a hand of a user. The position controller 41 can further include a plurality of grips 47 on lateral edges of the position controller 41 to assist a user in manipulating the position controller 41.

In certain embodiments, the working channel can be utilized to deliver instruments, fluids, medications, implants, or other materials into an interior body region. The steerable working channel device 40 can be positioned in at least one other of the plurality of lumen (35) of the medical introducer 20 so that the separate steerable working channel device 40 and the imaging device 60 are independently controllable. In some embodiments, the working channel device 40 can be disposable and intended for a single use.

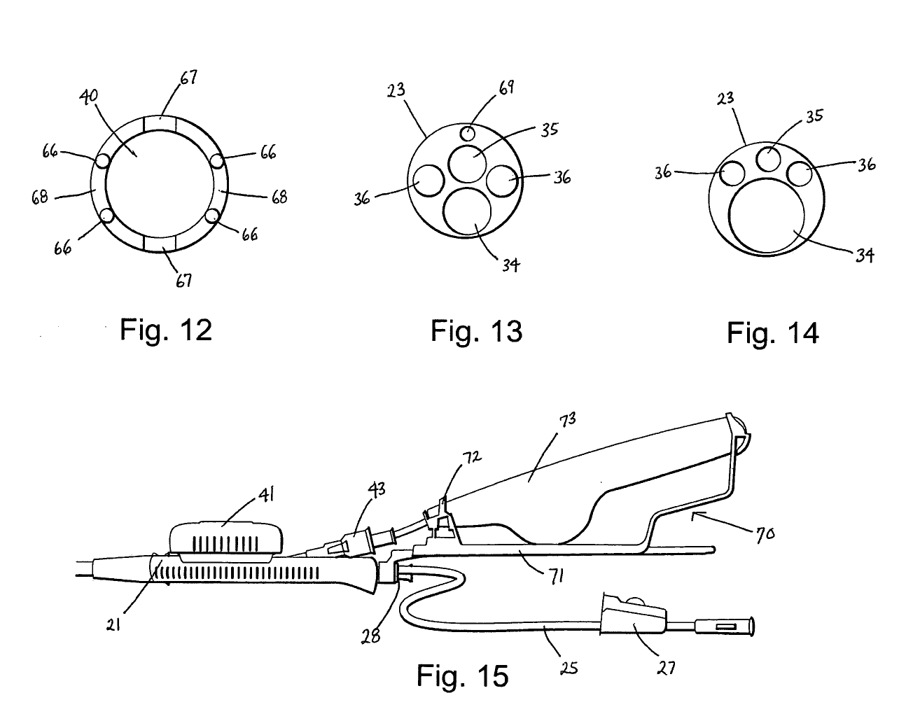

An example of an embodiment of a flexible distal portion 12 and steering wire configuration is shown in FIG. 12. In this embodiment, the working channel tube of the steerable working channel device 40 can comprise a proximal 11 insertion portion, a distal portion 12, and a distal tip 13. The proximal 11 insertion portion can be formed of a semi-rigid material 67, for example, pellethane having a 75 durometer hardness rating. The distal portion 12 can be formed of a combination of a relatively harder material 67, such as a 75 durometer pellethane, and a relatively softer, flexible material 68, such as pellethane having a 55 durometer. The portion of the distal portion 12 having different relative hardness can be co-extruded. The distal tip 13 can be formed of a semi-rigid material 67, which can be the same material from which the proximal 11 insertion portion is formed (for example, pellethane having a 75 durometer). In certain embodiments, other materials can be used to form the elongate tube 42 of the steerable working channel device 40.

The working channel tube 42 can include at least one steering lumen 66 in each lateral aspect of the tube 42. The steering wires can be routed from the position controller 41 through the steering wire lumens 66 through the flexible distal portion 12 and attached to the distal tip 13. The distal tip 13 is preferably formed of a harder material 67, such as a 75 durometer Pellethane, to provide a strong and firm anchor for the small diameter stainless steel steering wires that may cut through a softer material 68 when retracted. The flexible distal portion 12 can include a relatively softer material 68 in each of the lateral aspects through which the steering wire lumens 66 are formed, and a relatively harder material 67 in the dorsal and ventral aspects of the distal portion 12 tubing. Such a configuration can permit the distal portion 12 to deflect in a predetermined manner and amount. The presence of the relatively harder material 67 in the distal portion 12 allows the relatively softer, lateral sections 68 to deflect without compressing when extreme deflection is occurring, which can result in exposing an instrument in the steerable working channel more than desired. Different relative durometers of material can be utilized to achieve a relative hardness/softness ratio between sections of the distal portion 12 so as to allow directionally-controlled deflection of the distal portion 12 of the working channel tube 42.

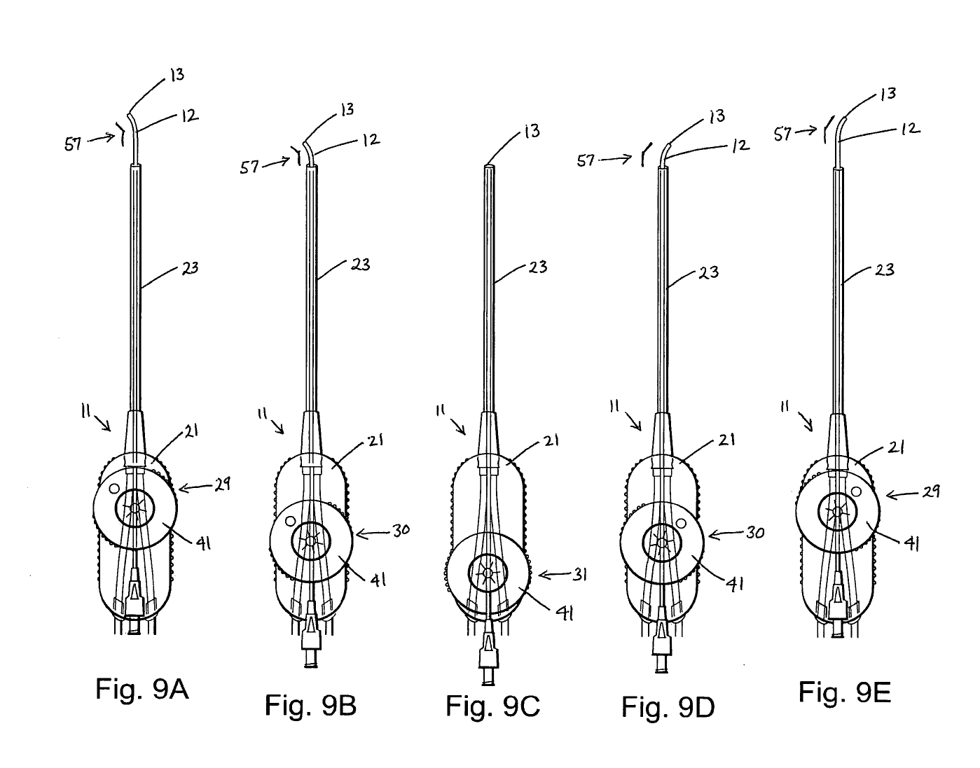

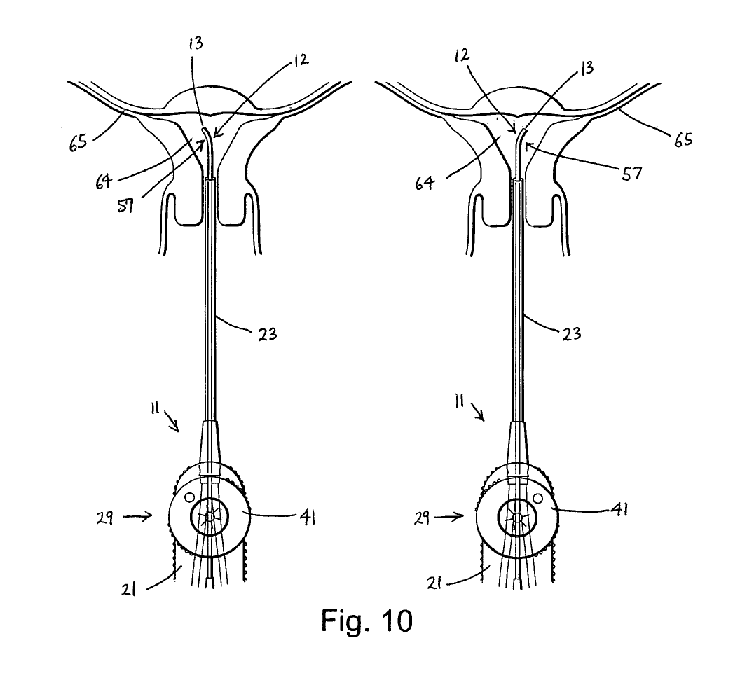

When the position controller upper housing portion 50 is rotated, one of the steering wires connected to the rotor 55 is wound about the rotor 55, causing the distal end of that steering wire to retract. This retraction pulls on the lateral side of the distal tip of the working channel tube 42 to which it is connected so as to “deflect” the distal tip and distal portion 12 at an angle 57 laterally away from the longitudinal axis 33 of the working channel tube 42, as shown in FIGS. 9 and 10. The position controller upper housing 50 can be rotated in the opposite direction to place tension on, or retract, the other steering wire and thereby “deflect” the distal portion 12 of the working channel tube 42 in the opposite direction. The position controller 41 can thus control the angular attitude of the distal portion 12 of the working channel tube 42. The steering wires in cooperation with the position controller 41 can be configured to limit angular adjustments of the distal portion 12 to a plane extending generally parallel to the upper surface of the position controller 41. For example, the configuration of the position controller 41 and the steering wires can be such that angular deflection 57 of the distal portion 12 of the working channel tube 42 can be limited to no more than 30 degrees, 45 degrees, or another predetermined limit. In other embodiments, various other steering mechanisms, such as one or more position deflectors associated with the working channel tube 42, can be used in accordance with the present invention.

In some embodiments, the position controller 41 can include a braking mechanism (not shown) for securing the upper and lower housing portions 50, 51, respectively, into position relative to each other. The braking mechanism can comprise, for example, a soft polymeric material, such as silicone, coated onto the outer surface(s) of the upper housing rotor 55 and/or the lower housing hub 54. In this fashion, the coated surface can allow the rotor 55 to rotate smoothly within the hub 54, while providing sufficient friction to hold the rotor 55 and the hub 54 of the upper and lower housings 50, 51, respectively, in position when released by an operator. In certain embodiments, in addition to providing a polymeric coating on the rotor 55 and/or hub 54 outer surfaces, one or both of these surfaces can be textured so as to provide further friction and greater securing force between the rotor 55 and hub 54. Such a braking mechanism is simple, inexpensive, and avoids any need for stronger mechanical or gear-based braking mechanisms. In particular embodiments, such a polymeric coating braking mechanism can be combined with other braking means.

As will be appreciated, a braking mechanism, of fixing in an alternate manner, in some embodiments, advantageously allows a predetermined route of delivery to be established. An advantageous result is increased precision and reduced time for procedures. Further, in some embodiments, the steerable working channel may be fixed prior to insertion into a patient.

In certain embodiments, for example, those that include a polymeric coating on the outer surfaces of the rotor 55 and hub 54, the internal brake mechanism can hold position automatically when steered to a particular point. This feature provides a physician with a precise control that is maintained when her/his fingers are removed from the position controller 41, for example, to perform another task during a procedure.

The position controller 41 can be adapted to control movement (extension and retraction) of the working channel tube 42 in the proximal and distal directions. In some embodiments, the inside of the medical introducer handle 21 can include detents (not shown) at various stop points along the length of the handle 21. For example, the medical introducer handle 21 can include a detent at a proximal position 31, medial position 30, and a distal position 29 on the inside of the handle 21. The bracket 52 and securing flange 53 on the lower side of the position controller 41 can slide along the length of the handle 21. When the securing flange 53 reaches a detent, the securing flange 53 engages the detent so as to secure the position controller 41 in that position. In this manner, as shown in FIG. 9, the position controller 41 can be moved in the proximal and distal directions and snap fit into detents at respective proximal 31, medial 30, and distal 29 positions in the handle 21 so as to control the distance the distal tip 13 of the working channel tube 42 extends beyond the distal tip 13 of the introducer tube 23 of the medical introducer 20. In certain embodiments, the proximal end 31 of the medical introducer handle 21 can include a recess to allow clearance for the working channel tube 42 extending from the proximal end 11 of the steerable working channel device 40, the connection port 38, and any attached accessories to slide the entire length of the handle 21 in the proximal direction.

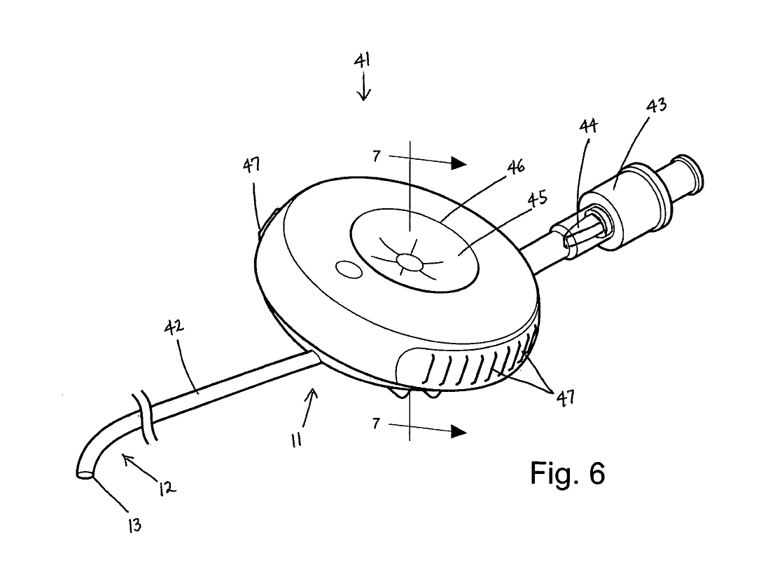

The position controller 41 can have a size adapted to fit between the fingers and thumb of an operator. In some embodiments, for example, as shown in FIGS. 6 and 7, the center of the position controller 41 can include a thumb depression 45, designed to allow a physician to place a thumb in the depression 45 to move the position controller 41 in the proximal and distal directions along the length of the medical introducer 20. The upper surface of the upper housing 50 of the position controller 41 can include a circular ridge 46 about the center of the position controller 41. The circular ridge 46 can provide a physician an ability to locate the center of the position controller 41 by “feel” rather by having to look at the controller 41.

In some embodiments, as shown for example, in FIGS. 1, 6, and 8, the position controller 41 can include a plurality of raised ridges, or grips, 47 on the lateral side edges of upper housing 50. The grip 47 surfaces can include a soft, tactile material that can provide improved grip and performance with the position controller 41. Such grips 47 can provide a positive grip on the upper housing 50 for rotating the upper housing 50 in the process of deflecting the distal portion 12 of the working channel tube 42.

The steering mechanism can provide the physician sufficient control of the distal tip 13 of the elongate tube 42 of the steerable working channel device 40 so as to manipulate the distal tip 13 of the working channel tube 42 for specific isolation on particular sections of an interior body region. The steering mechanism can allow the physician to steer the working channel tube 42 while simultaneously providing access to a lumen within the steerable working channel for inserting and using various surgical instruments and fluids. That is, the steering mechanism can provide the control and manipulation of the distal tip 13 the working channel tube 42 of the steerable working channel device 40 needed for use with the surgical instruments and fluids required for a procedure.

Another feature of some embodiments is that the insertion depth of the working channel device may be set to a predetermined value using the mechanisms described herein for steering and fixing the working channel.

In some embodiments, the steerable working channel device 40 as well can be controllable independent of the imaging device 60 positioned in the medical introducer 20 and independent of the medical introducer 20. Such a system can be used in a variety of medical procedures, including, for example, gynecological, fertility, hysteroscopy, or prostate type applications. For example, the medical device introduction system 10 and medical introducer 20 can be advantageously utilized in procedures and products related to insemination, profusion, intrauterine blastocyst/embryo transfer, endoscopic evaluation and operations, laproscopy (that is, culdoscopy, transvaginal hydro laparoscopy), and/or falloscopy. Accordingly, both fluid management and medical instruments usage may be a managed through the working channel device 40 independent of or separate from both the imaging device 60 and the medical introducer 20.

In certain embodiments, the separate working channel device 40—insertable through a separate lumen 35 in the medical introducer 20 from the lumen 34 in which the imaging device 60 is inserted—can be a non-steerable working channel device 40. In such an embodiment, the working channel device does not have a steering mechanism associated with the device 40. However, the non-steerable working channel device can be moved in the distal and proximal directions within one of the lumen 35 of the medical introducer 20.

In some embodiments of the separate working channel device 40, the proximal end 11 of the working channel tube 42 can include one or more access ports 38, as shown in FIGS. 1 and 6. Such access ports 38 can be sealed with a port seal 39. Such a seal 39 can be formed of an elastomeric material such as silicone rubber and have a very small axial opening through the material that permits a small object such as a needle to enter, but which otherwise prevents fluid flow in either direction, and thus protects the lumens from receiving contaminating materials therein. In some embodiments, the proximal access 38 on the working channel tube 42 can comprise a luer lock fitting and seal 44 for controllable access to the steerable working channel.

In some embodiments, the imaging device 60 can include an endoscopic cannula 62, a light delivery mechanism, and a imaging system. The imaging system can include at least one of an optical scope, an ultrasound instrument, and/or a camera 61. A camera may be positioned on a distal 12 portion of the endoscopic cannula 62.

In some embodiments, the introducer handle 21 can further include a scope connector 28 located on an opposite side of the handle 21 from the introducer tube 23 and longitudinally aligned with the one (34) of the plurality of lumens in the introducer tube 23. In this manner the imaging device 60 can be securely connected to the scope connector 28. In this configuration, that is, when the imaging device 60 is securely connected to the scope connector 28, the imaging device 60 can rotate independent of movement of the medical introducer 20.

In some embodiments, the endoscopic cannula, or endoscope, 62 can be rigid. In other embodiments, the endoscope 62 can be flexible. An embodiment of a flexible endoscopic cannula 62 can include a proximal 11 portion having a first durometer and a distal 12 portion having a second durometer. The second durometer is lower than the first durometer, which can allow deflection of the distal portion 12 for improved viewing of a target area in the interior body region. Some embodiments of the imaging device 60 can further include at least two steering wires (not shown), each wire having its distal end connected to the distal tip 13 of the endoscopic cannula 62. The steering wires can extend at least the length of the endoscopic cannula 62. The proximal end of the steering wires can be operably connected to a deflection control mechanism at the proximal end 11 of the endoscopic cannula 62. In this way, actuation of the deflection control mechanism can cause the distal tip 13 of the endoscopic cannula 62 to deflect at an angle away from the longitudinal axis 33 of the imaging device 60. The endoscopic cannula 62 can include each of a first pair of wires adjacent opposite points on a circumference of the endoscopic cannula 62 to deflect the distal tip along a first axis. The endoscopic cannula 62 can also include each of a second pair of wires adjacent two other opposite points on the circumference of the endoscopic cannula 62. Each of the second pair of wires can be positioned 90 degrees from each of the first pair of wires, to deflect the distal tip along a second axis perpendicular to the first axis.

In some embodiments, the light delivery mechanism can include one or more light emitting diodes (not shown) mounted at a distal tip of the endoscopic cannula 62. In other embodiments, the light delivery mechanism can include a plurality of light delivery fibers (not shown) attached to the endoscopic cannula 62 and extending from the proximal end 11 to the distal tip 13 of the endoscopic cannula 62. The light delivery mechanism can further include a light source (not shown) comprising a light cable attached on one end to a power source and on the opposite end to the light delivery fibers at the proximal end 11 of the endoscopic cannula 62. Alternatively, the light delivery mechanism can further include a light source comprising one or more light emitting diodes connected to the light delivery fibers at the proximal end 11 of the endoscopic cannula 62. In another embodiment, the light delivery mechanism can include a plurality of light delivery fibers integrated into the endoscopic cannula 62 that extend from the proximal end 11 to the distal tip 13 of the endoscopic cannula 62. In this embodiment, the light delivery mechanism can further include a light source comprising a light cable attached on one end to a power source and on the opposite end to the light delivery fibers at the proximal end 11 of the endoscopic cannula 62. Alternatively, the light delivery mechanism can further include a light source comprising light emitting diodes in the introducer handle connected to the light delivery fibers.

In some embodiments, the medical device introduction system 10 of the present invention can include an imaging device 60. The imaging device 60 can be separate from the medical introducer 20, and can be positioned in a predetermined one (34) of the plurality of lumens of the medical introducer 20, for example, in the dedicated scope lumen 34. The scope lumen 34 can be configured to receive various types of imaging devices 60 therein. The imaging device 60 can be removably connected to the medical introducer 20.

As described herein, in various embodiments of the medical device introduction system 10, the imaging device 60 can be operated independent of the medical introducer 20 and/or the working channel device 40, thereby permitting a steady, or constant, view of a particular anatomical structure or site in an interior body region while the introducer 20 and/or the working channel device 40 are manipulated. Such an independent operation of the imaging device 60 can be accomplished, for example, through cooperation of the imaging device 60 with the scope port, or connector, 28 as shown in FIGS. 1-3.

The scope connector 28 is fixed to, for example, by being integrally molded with, the proximal end 14 of the medical introducer handle 21. The scope connector 28 can be positioned in longitudinal alignment with the dedicated scope lumen 34 in the introducer manifold 22. The scope connector 28 can include a molded luer lock fitting, which allows the scope 62 to be securely connected to the introducer handle 21, and to also rotate about its longitudinal axis 33 independent from movement of the medical introducer 20. In an application in which the scope 62 is not secured to the introducer handle 21, the imaging device 60 can also be rotated about its longitudinal axis 33 independent from movement of the medical introducer 20. In this way, the medical introducer 20 and/or the working channel device 40 associated therewith can be moved without moving the imaging device 60. As a result, the view through the imaging device 60 can remain constant, providing a fixed reference point for movement of the introducer 20 and/or working channel device 40, and thereby allowing the physician to maintain a steady, right-side-up orientation of view and movement in the interior body region.

The imaging device 60 can comprise, for example, an optical scope, such as a fiberoptic scope, a camera 61, a charge couple device (CCD), a camera positioned on the distal tip 13 and/or distal portion 12 of an elongate shaft (62), known as a “chip-on-a-stick,” or ultrasound or other sonic device. The imaging device 60 can include a light source (not shown) for illuminating an interior body region. The light source can be separate from, and removably connected to, the imaging device 60. Alternatively, the light source can be integrated with the imaging device 60. As shown in the embodiment in FIGS. 1, 3, and 11, the imaging device 60 can include a fiberscope 62 operably connected to an ocular mechanism, such as an endoscope lens 63, to adjust focus or light intensity. The fiberscope 62 can be, for example, a 2.0 mm 50 K fiberscope, and the endoscope lens 63 can be a 2.9 mm 30 degree rod lens. As shown in this embodiment, the imaging device 60 can be a “low profile” camera 61, which is less bulky, weighs less, and more easily maneuverable than other cameras, and is configured to readily cooperate with other components of the medical device introduction system 10.

The imaging device 60 can be connected to a monitor or other display mechanism for viewing an image within at least a portion of the interior body region into which the imaging device 60 is inserted. The imaging device 60 can be connected to an image capture mechanism, for example, a computer-readable medium such as a computer hard drive, a memory stick, a compact disc, a digital versatile disc, magnetic tape, or other storage medium, for recording images viewed via the imaging device.

Embodiments of medical device introduction systems and methods of the present invention provide advantages over conventional systems and methods. The cooperation of the medical introducer 20, related to, for example, the modular introducer handle 21 and introducer tube 23 and fluid delivery in dedicated lumens 36; the separate steerable working channel device 40, including ease of introduction of accessory devices and precision of device positioning and utilization through the working channel; the separate imaging device 60 delivered through a dedicated lumen 34; and the control of each of the medical introducer 20, steerable working channel device 40, and imaging device 60 independent of each other device provide for effectiveness of operation.

Such medical device introduction systems and methods of the present invention can allow a physician, or other medical personnel, to control and manipulate the working channel device 40, an imaging source 60, and other medical devices inserted into an interior body region through the medical introducer 20, while simultaneously using surgical tools and fluids needed for such procedures. In this manner, the physician may be allowed to positionally locate, isolate, and view problem areas with greater precision within the interior body region than with conventional medical device introduction systems and methods. That is, control of visualization, access, and use of instrumentation in the operative site environment can be enhanced by the cooperation of the various combinations of components as described herein. In part due to the simple design, embodiments of the present invention can be easy to use and thus may require minimal training. Such factors can allow a physician to utilize embodiments of the present invention to perform procedures in an office setting which may have previously been avoided due to complexity and cost.

In particular, the ability to maintain a constant, or fixed, point of reference, for example, by keeping the imaging device 60 steady while re-positioning the medical introducer 20 and/or the working channel device(s) 40 can provide greater control over the medical procedure, and may decrease operative time. Embodiments of medical device introduction systems 10, devices 20, kits, and methods of the present invention can be utilized in conjunction with procedures that are minimally invasive. Whether used alone or in the context of minimally invasive procedures, embodiments of the present invention can advantageously provide, for example, performing the procedure on an outpatient basis, reduced trauma to the target area, reduced anesthesia time, reduced recovery time, and decreased discomfort to the patient. As an example, in a hysteroscopy system, an embodiment of the present invention can allow a fixed endoscope 62 position, thereby minimizing tissue trauma as compared to conventional hysteroscopy procedures. In addition, minimal outside diameters of the medical introducer 20 and associated components resulting in smaller devices can decrease the need for anesthesia and can increase patient comfort related to a procedure.

Single use components can be safer than reusable devices due to the decrease or elimination of risk for transmission of communicable infections and diseases between patients. Single use components can be more cost-effective due to elimination of cleaning and sterilization expense and decreased expense for repairs associated with reusable devices

In another aspect of the present invention, certain embodiments of the medical introducer 20 can further include a lift wire not shown) attached on its proximal end to a distal tip lift control (not shown), such as a knob similar to the steerable working channel device position controller 41. The lift wire can be routed through a dedicated lift wire lumen 69, as shown in FIG. 13, through the length of the medical introducer tube 23 and attached on its distal end to the distal tip 13 of the introducer tube 23. The distal lift control can be moved in the proximal direction so as to pull the lift wire in the proximal direction, thereby deflecting the distal tip 13 of the introducer tube 23 in one direction. When the distal tip 13 of the introducer tube 23 is lifted, any device therein will also be lifted, or deflected, along with the introducer tube 20. In operation, the introducer tube 20 can be inserted in the straight position (along its longitudinal axis).

In an exemplary embodiment, a flexible medical device, such as a flexible hysteroscope, can be inserted in the working channel, or lumen, 35 of the medical introducer 20. Once the introducer tube 23 is inserted in the straight position into the uterine cavity 64 (FIG. 10) and the cavity 64 distended, the distal tip lift control can be moved in the proximal direction so as to lift the distal tip of the introducer tube 23 in one direction. The introducer 20 can then be rotated to view the extreme left and right aspects of the uterine cavity 64. The distal tip of the introducer tube 23 can be further positioned and aligned with the tubal os for delivery of an instrument or implant to the fallopian tube 65. Such an embodiment can thus provide a simple operation for lifting, or deflecting, a steerable working channel device 40, imaging device 60, or other medical device in an interior body region.

Some embodiments of a medical device introduction system 10 of the present invention can include an accessory device support 70, as shown in FIG. 15. The accessory device support 70 can be removably connected to the introducer handle 21. The accessory device support 70 can comprise a carrier arm 72 for supporting an upper part of a body of a separate medical device 73 to be used with the medical introducer 20, and a slide member (or mechanism) 71 for slidably supporting a lower part of the body of the separate medical device 73. This accessory device support 70 can be used to stabilize placement of additional separate medical devices (73) in the interior body region. In certain embodiments, the accessory device support 70 can be removably connected to the outside surface of the scope connector 28 on the proximal end of the introducer handle 21.

An embodiment of the present invention can include a delivery catheter having a small delivery channel, or working lumen 35, as shown in FIG. 14. Such a configuration allows the scope lumen 34 to be larger than, for example, the embodiment shown in FIG. 5. In the embodiment shown in FIG. 14, the catheter can be inserted into the interior body region in the straight position. For example, a flexible hysteroscope can be introduced into the uterine cavity 64 in the straight position via a small delivery catheter. Once inserted, and the cavity 64 is distended, the medical introducer 20 can be rotated to provide an optimal viewing angle. The flexible hysteroscope can have a pre-formed “angle up” distal tip 13, and can be inserted via the working delivery channel in an obturator. Once in the uterine cavity 64, the obturator can be removed, and the angled distal tip is restored for use. This enables a zero degree angle of view flexible scope to be utilized and a more effective access approach to particular pathologies. Such a small diameter delivery catheter can assist visualization and access in difficult to reach pathology. In addition, a small diameter catheter can improve patient comfort relative to larger delivery catheters.

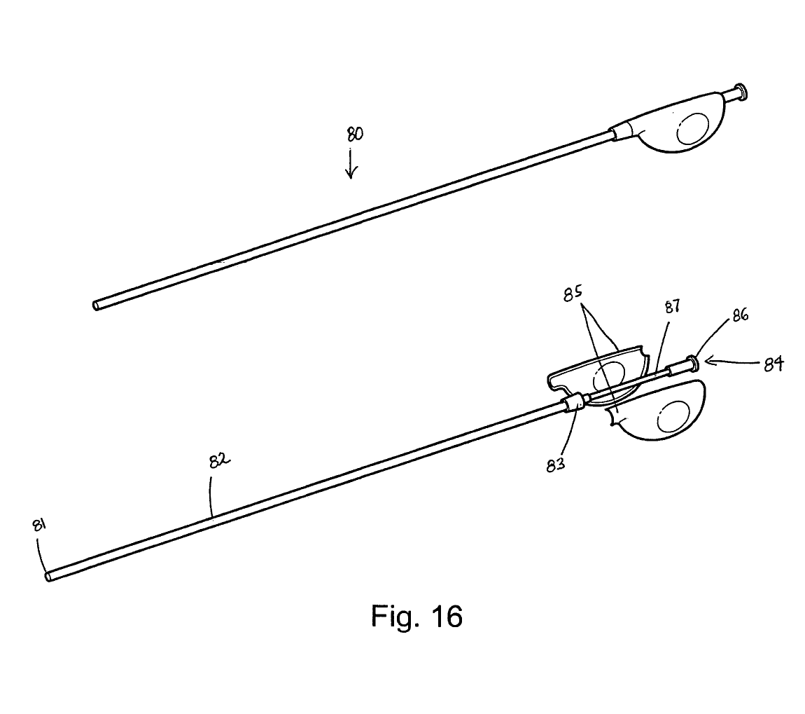

As shown in FIG. 16, an embodiment of the present invention can include a continuous flow examination sheath 80. This device 80 can be single-use and utilized for quick evaluation or hysteroscopy, for example. The continuous flow examination sheath 80 can include a formed distal tip 81, an insertion portion 82, a fluid-out adapter 84, a fluid-in adapter 83, a finger grip 85, a proximal port 86, and an inner sheath 87. An endoscope 62 can be inserted through the proximal port 86 through a fluid seal adapter (not shown). The fluid-in tube 83 can allow a physician to deliver fluid to clear the scope 62 lens or distend the uterine cavity 64 for improved visualization. In addition, the fluid-out adapter 84, and tube, can allow the physician to clear fluid from the cavity 64 that may impair viewing caused by blood present at the site.

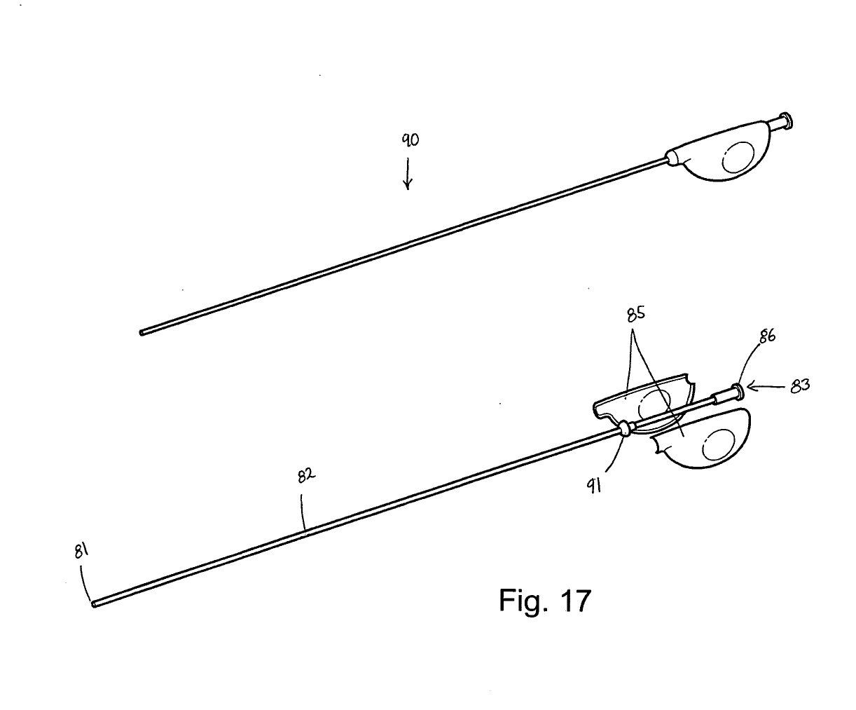

As shown in FIG. 17, an embodiment of the present invention can include a single flow examination sheath 90. This device 90 can be single-use and utilized for quick evaluation or hysteroscopy, for example. The single flow examination sheath 90 can include a formed distal tip 81, an insertion portion 82, a fluid-in adapter 83, a finger grip 85, a proximal port 86, and a nose piece 91. An endoscope 62 can be inserted through the proximal port 86 through a fluid seal adapter. The fluid-in tube 83 can allow a physician to deliver fluid to clear the scope lens or distend the uterine cavity 64 for improved visualization.

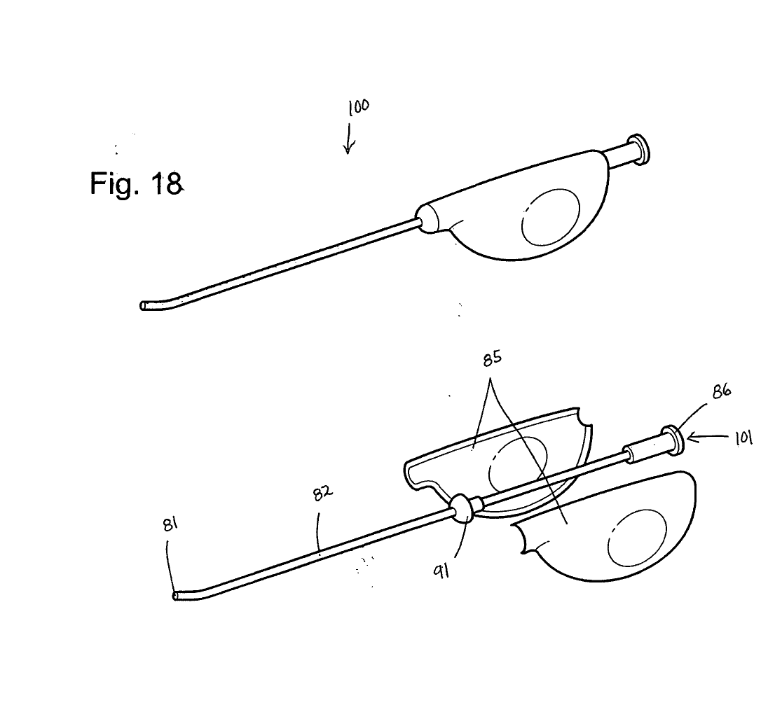

As shown in FIG. 18, an embodiment of the present invention can include a pre-formed delivery catheter 100. The pre-formed delivery catheter 100 can include a formed distal tip 81, an insertion portion 82, an adapter 101, a finger grip 85, a proximal port 86, and a nose piece 91. This device 100 can be used for delivering another medical device or treatment to a specific site when a steerable mechanism is not practical. Fluid can be incorporated by adapters known in the art, for example, a Touhy Borst adapter and a side port entry attached to the proximal end of the catheter 100.

In another embodiment, an endoscopy system utilized in the present invention can be a wireless handheld endoscopy system (not shown). Such a system can include an endoscopic cannula 62, a disposable mount, a focus/zoom function, a wireless camera, for example, a 2.4 GHz, high resolution camera used in cooperation with a laptop or other monitor, and controls for imaging and power.

Some embodiments of a medical device introduction system 10 can be utilized with a conventional endoscope trocar system (not shown), for example, for abdominal minimally invasive surgery. The medical introducer 20 can be inserted through a 10 mm or 5 mm trocar and can be sealed by the internal trocar seal. When inserted with a conventional trocar system, embodiments of the present invention can retain all functionality described herein, including depth adjustment for the medical introducer 20, 360 degrees of rotation, depth adjustment for the steerable working channel device 40, and angle and direction of deflection adjustment, visualization, and access related to the working channel device 40.

Some embodiments of the present invention can include a kit comprising one or more of various components of a medical device introduction system 10, including a medical introducer 20, a separate imaging device 60, and/or a separate working channel device 40. The medical introducer 20 can include a handle 21 and an elongate introducer tube 23 extending from the distal end 15 of the handle 21. The introducer tube 23 can include a plurality of lumens 34, 35, 36 extending longitudinally therein. The medical introducer 20 may be inserted into an interior body region of a patient. The separate imaging device 60 may be inserted through the handle 21 and positioned in a predetermined one (34) of the plurality of lumens. The imaging device 60 can have an interface with the handle 21 such that each of the imaging device 60 and the medical introducer 20 is movable independent of the other. The separate working channel device 40 can include an working channel tube 42 and a position controller 41. The working channel tube 42 can include at least one lumen extending the length thereof defining a working channel. The position controller 41 can be configured to control positioning of the working channel tube 42. The working channel device 40 may be removably connectable to the handle 21 and positioned in another predetermined one (35) of the plurality of lumens. In some embodiments of a kit of the present invention, each of the medical introducer 20, the imaging device 60, and the working channel device 40 can be movable independent of the other.

In certain embodiments, the medical introducer handle 21 can comprise an oval-shaped ring of material having an open interior. The handle 21 can have a proximal end 14 configured to receive at least one fluid tube 24, 25 and the imaging device 60 therethrough. The handle 21 can further include a distal end 15 adapted to connect to the introducer tube 23. In certain embodiments, the plurality of lumens in the introducer tube 23 can include a scope lumen 34, at least one working lumen 35, and at least one fluid lumen 36 separate from the scope lumen 34 and the working lumen(s) 36. In an illustrative embodiment, the medical introducer 20 can further include a fluid inflow tube 24 routed through the proximal end 14 of the handle 21 and in fluid communication with a fluid lumen 36, and a fluid outflow tube 25 routed through the proximal end 14 of the handle 21 and in fluid communication with another fluid lumen 36.

In some embodiments, the medical introducer 20 can include a modular manifold 22 integrally formed on the proximal end 11 of the introducer tube 23 and have a corresponding plurality of lumens 34, 35, 36 aligned with the plurality of lumens 34, 35, 36 in the introducer tube 23. The manifold 22 can be removably connected to the introducer handle 21 such that the manifold 22 and introducer tube 23 are interchangeable in the handle 21 with other manifolds 22 and introducer tubes 23. In particular embodiments, a kit can include a plurality of manifolds 22 and introducer tubes 23, such that one manifold 22 and introducer tube 23 in a kit may be interchanged on a handle 21 with another one of the manifolds 22 and introducer tubes 23 in the kit.

In some embodiments, the medical introducer 20 and/or the working channel device 40 can be disposable. In some embodiments, at least a portion of the medical introducer 20 and/or at least a portion of the working channel device 40 can be translucent such that passage of materials therethrough is viewable.

In some embodiments, one or more of the introducer tube 23, the working channel tube 42, and the endoscopic cannula 62 can include a proximal 11 portion having a first durometer and a distal 12 portion having a second durometer. The second durometer is lower than the first durometer so as to allow deflection of the distal 12 portion of the respective tube or cannula for controllable access to a target area in the interior body region.

In certain embodiments, the working channel device 40 can be a steerable working channel device 40. In such an embodiment, the working channel tube 42 can comprise a flexible distal portion 12 for steering to selected positions. The position controller 41 can be operably connected to the working channel tube distal 12 portion and slidable within the introducer handle 21 for moving the working channel tube distal 12 portion in distal and proximal directions. In addition, the position controller 41 be actuated to steer the flexible distal 12 portion of the working channel tube 42 in predetermined directions and amounts.

In some embodiments, the imaging device 60 can include an endoscopic cannula 62, a light delivery mechanism (not shown), and a imaging system. The light delivery system can comprise light emitting diodes and/or light delivery fibers. The imaging system can be an optical scope 62, an ultrasound instrument, or a camera 61.

In certain embodiments, a kit can include other devices and/or instruments that may be used with the medical device introduction system 10. For example, such a kit may include an accessory device support 70 removably connectable to the outside surface of a scope connector 28 on the proximal end 14 of the introducer handle 21. The accessory device support 70 can comprise a carrier arm 72 for supporting an upper part of a body of a separate medical device 73 to be used with the medical introducer 20 and a slide member 71 for slidably supporting a lower part of the body of the separate medical device 73. Such an accessory device support 70 may be used to facilitate and stabilize placement of a separate medical device 73 in the interior body region.

The present invention can include embodiments of a method. For example, a medical introducer 20 comprising a handle 21 and an introducer tube 23 extending therefrom and having a plurality of lumens 34, 35, 36 extending longitudinally therein can be inserted into an interior body region of a patient. A separate imaging device 60 can be inserted through the handle 21 and in a predetermined one of the plurality of lumens 34, 35, 36. The imaging device 60 can be positioned in a selected position within the interior body region. Then, an image can be produced from within the interior body region. A separate working channel device 40 and position controller 41 can be removably connected to the medical introducer 20. The working channel device 40 can include an working channel tube 42 having at least one lumen extending the length thereof defining a working channel. The position controller 41 for controlling the position of the working channel tube 42 can be positioned in the working channel in another predetermined one (35) of the plurality of lumens. In such embodiments, one of the group of the medical introducer 20, the imaging device 60, and the working channel device 40 may be moved independently of the others of the group.

In some embodiments of a method, the medical introducer handle 21 can comprise an oval-shaped ring of material having an open interior. The method can further include connecting a distal end of the handle 21 to the introducer tube 23. In some embodiments of a method, the medical introducer 20 can include a modular manifold 22 integrally formed on a proximal end 11 of the introducer tube 23 and have a corresponding plurality of lumens 34, 35, 36 aligned with the plurality of lumens 34, 35, 36 in the introducer tube 23. In such an embodiment, the manifold 22 can be removably connected to the introducer handle 21. The manifold 22 and introducer tube 23 may be interchanged in the handle 21 with other manifolds 22 and introducer tubes 23.

Certain embodiments of a method of the present invention include performing a medical procedure in an interior body region through the working channel device 40. For example, the medical procedure can be a gynecological procedure, a spinal procedure, or other procedure.

In some embodiments, a kit comprises at least one of a medical introducer; an imaging device; or a working channel device. In some embodiments a kit comprises a medical introducer and a working channel device. In some embodiment a kit comprises a working channel device inserted into a medical introducer.

The devices, systems, kits, and methods embodying the present invention can be adapted for use in many suitable interior body regions in humans and animals, wherever it may be desirable to provide support for a tissue. The illustrative embodiments are described in association with devices, systems, kits, and methods used to access interior body regions such as the uterine cavity 64. For example, the medical device introduction system 10, and, in particular, the cooperating medical introducer 20, steerable working channel device 40, and imaging device 60 can be utilized to perform a hysteroscopy.

Some embodiments of the present invention may be utilized in applications other than those described herein. In some embodiments, the present invention may be used in other interior body regions or types of tissue. For example, certain embodiments of a medical device introduction system 10 of the present invention can be adapted for use in procedures related to the spinal column, for example, in the epidural space. In a particular embodiment, for example, the medical device introduction system according to the present invention may be utilized in an upright ventral epiduroscopic laser discectomy, in which the procedure is performed with the patient in an upright, symptomatic position such that diagnosis and treatment can be performed interactively with axial loading pressure on the affected intervertebral disc.

Features of a medical device introduction system and method of the present invention may be accomplished singularly, or in combination, in one or more of the embodiments of the present invention. Although particular embodiments have been described, it should be recognized that these embodiments are merely illustrative of the principles of the present invention. Those of ordinary skill in the art will appreciate that a medical device introduction system 10 and method of the present invention may be constructed and implemented in other ways and embodiments. Accordingly, the description herein should not be read as limiting the present invention, as other embodiments also fall within the scope of the present invention.

What is claimed is:

1. A medical device introduction system, comprising:
a medical introducer comprising a handle and an elongate introducer tube extending therefrom and having a plurality of lumens extending longitudinally therein, the medical introducer insertable into an interior body region of a patient;
a separate imaging device insertable through the handle and positionable in a predetermined one of the plurality of lumens, the imaging device having an interface with the handle such that each of the imaging device and the medical introducer is movable independent of the other; and
a separate working channel device comprising an elongate working channel tube having at least one lumen extending the length thereof defining a working channel and a position controller for controlling the position of the working channel tube, the working channel device removably connectable to the handle and positionable in another predetermined one of the plurality of lumens such that each of the medical introducer, the imaging device, and the working channel device is movable independent of the other.

2. The system of claim 1, wherein the medical introducer handle comprises an oval-shaped ring of material having an open interior, a proximal end configured to receive at least one fluid tube and the imaging device therethrough, and a distal end adapted to connect to the introducer tube.

3. The system of claim 1, wherein the plurality of lumens further comprises a scope lumen, at least one working lumen, and at least one fluid lumen separate from the scope lumen and the at least one working lumen.

4. The system of claim 3, wherein the medical introducer further comprises a fluid inflow tube routed through the proximal end of the handle and in fluid communication with one of the at least one fluid lumen and a fluid outflow tube routed through the proximal end of the handle and in fluid communication with another one of the at least one fluid lumen.

5. The system of claim 3, wherein the diameter of the working lumen is larger than the diameter of the other lumens.

6. The system of claim 1, wherein the medical introducer further comprises a modular manifold integrally formed on a proximal end of the introducer tube and having a corresponding plurality of lumens aligned with the plurality of lumens in the introducer tube, the manifold removably connectable to the introducer handle such that the manifold and introducer tube are interchangeable in the handle with other manifolds and introducer tubes.

7. The system of claim 1, wherein the medical introducer is disposable.

8. The system of claim 1, wherein the working channel device is disposable.

9. The system of claim 1, wherein at least a portion of the medical introducer is translucent such that passage of materials therethrough is viewable.

10. The system of claim 1, wherein at least a portion of the working channel device is translucent such that passage of materials therethrough is viewable.

11. The system of claim 1, the introducer tube further comprising a proximal portion having a first durometer and a distal portion having a second durometer, wherein the second durometer is lower than the first durometer so as to allow deflection of the distal portion for controllable access to a target area in the interior body region.

12. The system of claim 11, wherein the introducer tube distal portion further comprises a distal tip having a first diameter smaller than a second diameter of the remainder of the introducer tube, the smaller first diameter adapted to seal about a device extending beyond the distal tip.

13. The system of claim 11, wherein the introducer tube further comprises a fluid lumen comprising a wall having a third durometer that is higher than the second durometer of the distal portion so as to prevent collapsing of the fluid lumen when the distal portion of the introducer tube is deflected.

14. The system of claim 1, wherein the handle further comprises a plurality of raised grips on an outside surface of the handle.

15. The system of claim 1, wherein the working channel device further comprises a steerable working channel device, and wherein the working channel tube comprises a flexible distal portion adapted for steering to selected positions.

16. The system of claim 15, wherein the position controller is operably connected to the working channel tube distal portion and slidable within the introducer handle for moving the working channel tube distal portion in distal and proximal directions.

17. The system of claim 16, wherein the position controller is actuatable to steer the flexible distal portion of the working channel tube in predetermined directions and amounts.

18. The system of claim 17, wherein the predetermined direction of steering comprises a plane generally parallel to an upper surface of the position controller.

19. The system of claim 16, wherein the steerable working channel device further comprises at least two steering wires each having a distal end connected to a distal tip of the working channel tube, extending through the working channel tube, and having a proximal end operably connected to the position controller to manipulate the distal portions of the working channel tube.

20. The system of claim 19, wherein the position controller further comprises a circular, lower housing having an upwardly extending hollow hub and a cooperating circular, upper housing having a downwardly extending rotor rotatingly seated inside the hollow hub, wherein each of the steering wires is connected to an opposite side of the position controller rotor such that rotation of the upper housing causes rotation of the rotor inside the hub and the distal end of the steering wire on one side of the rotor to retract so as to deflect the distal tip at an angle laterally away from a longitudinal axis of the working channel tube.

21. The system of claim 20,
wherein the introducer handle further comprises an oval-shaped ring of material having an open interior and a plurality of detents on an inner surface of the handle from a proximal position to a distal position,
wherein the lower housing of the position controller further comprises a downwardly extending bracket adapted to friction fit in the inner surface of the handle and a securing flange extending outwardly from the bracket adapted to friction fit about a bottom of the handle,
wherein the position controller is slidingly engageable with the detents so as to secure the position of the working channel tube distal tip along a longitudinal axis of the working channel tube.

22. The system of claim 20, wherein the position controller further comprises an automatic braking mechanism comprising a soft material on the outer surface of at least one of the upper housing rotor and the lower housing hub so as to provide sufficient friction to hold the upper housing in position relative to the lower housing when released by a user.

23. The system of claim 1, the working channel tube further comprising a proximal portion having a first durometer and a distal portion having a second durometer, wherein the second durometer is lower than the first durometer so as to allow deflection of the distal portion for improved access to a target area in the interior body region.

24. The system of claim 23, wherein the working channel tube distal portion further comprises a distal tip having a first diameter smaller than a second diameter of the remainder of the working channel tube, the smaller first diameter adapted to seal about a device extending beyond the distal tip.

25. The system of claim 1, wherein the working channel device further comprises at least one access port having a seal and connected to a proximal end of the working channel tube for controllable access to the steerable working channel.

26. The system of claim 1, wherein the position controller further comprises a size adapted to be readily held in a hand of a user.

27. The system of claim 1, wherein the position controller further comprises a plurality of grips on lateral edges of the position controller to assist a user in manipulating the position controller.

28. The system of claim 1, wherein the imaging device further comprises an endoscopic cannula, a light delivery mechanism, and a imaging system.

29. The system of claim 28, wherein the imaging system comprises at least one of an optical scope, an ultrasound instrument, or a camera positioned on a distal portion of the endoscopic cannula.

30. The system of claim 1, wherein the introducer handle further comprises a scope connector located on an opposite side of the handle from the introducer tube and longitudinally aligned with the one of the plurality of lumens in the introducer tube, and wherein the imaging device is securely connectable to the scope connector.

31. The system of claim 30, wherein the imaging device is adapted to rotate independent of movement of the medical introducer when the imaging device is securely connected to the scope connector.

32. The system of claim 28, wherein the endoscopic cannula comprises a proximal portion having a first durometer and a distal portion having a second durometer, wherein the second durometer is lower than the first durometer so as to allow deflection of the distal portion for improved viewing of a target area in the interior body region.

33. The system of claim 28, the imaging device further comprising at least two steering wires each having a distal end connected to a distal tip of the endoscopic cannula, extending at least the length of the endoscopic cannula, and a having proximal end operably connected to a deflection control mechanism at a proximal end of the endoscopic cannula, wherein actuation of the deflection control mechanism causes the distal tip of the endoscopic cannula to deflect at an angle away from a longitudinal axis of the imaging device.

34. The system of claim 33, wherein the at least two steering wires further comprise each of a first pair of wires adjacent opposite points on a circumference of the endoscopic cannula to deflect the distal tip along a first axis, and each of a second pair of wires adjacent two other opposite points on the circumference of the endoscopic cannula, each of the second pair of wires positioned 90 degrees from each of the first pair of wires, to deflect the distal tip along a second axis perpendicular to the first axis.

35. The system of claim 28, wherein the light delivery mechanism comprises one or more light emitting diodes mounted at a distal tip of the endoscopic cannula.

36. The system of claim 28, wherein the light delivery mechanism comprises a plurality of light delivery fibers attached to the endoscopic cannula and extending from a proximal end to the distal tip of the endoscopic cannula.

37. The system of claim 36, the light delivery mechanism further comprising a light source comprising a light cable attached on one end to a power source and on the opposite end to the light delivery fibers at the proximal end of the endoscopic cannula.

38. The system of claim 37, the light delivery mechanism further comprising a light source comprising one or more light emitting diodes connected to the light delivery fibers at the proximal end of the endoscopic cannula.

39. The system of claim 28, wherein the light delivery mechanism comprises a plurality of light delivery fibers integrated into the endoscopic cannula and extending from a proximal end to the distal tip of the endoscopic cannula.

40. The system of claim 39, the light delivery mechanism further comprising a light source comprising a light cable attached on one end to a power source and on the opposite end to the light delivery fibers at the proximal end of the endoscopic cannula.

41. The system of claim 40, the light delivery mechanism further comprising a light source comprising light emitting diodes in the introducer handle connected to the light delivery fibers.

42. The system of claim 1, further comprising an accessory device support removably connectable to the introducer handle and comprising a carrier arm for supporting an upper part of a body of a separate medical device to be used with the medical introducer and a slide member for slidably supporting a lower part of the body of the separate medical device for stabilizing placement of the separate medical device in the interior body region.

43. The system of claim 42, wherein the accessory device support is removably connectable to an outside surface of a scope connector on a proximal end of the introducer handle.

44. A medical introducer device, comprising:
a handle comprising an oval-shaped ring of material having an open interior, a proximal end, and a distal end;
an elongate introducer tube extending from the distal end of the handle and having a plurality of lumens extending longitudinally therein;
the proximal end of the handle configured to receive at least one fluid tube and an imaging device therethrough; and
the distal end of the handle adapted to connect to the introducer tube,
wherein the device is insertable into an interior body region of a patient.

45. The device of claim 44, wherein the plurality of lumens further comprises a scope lumen, at least one working lumen, and at least one fluid lumen separate from the scope lumen and the at least one working lumen.

46. The device of claim 45, further comprising a fluid inflow tube routed through the proximal end of the handle and in fluid communication with one of the at least one fluid lumen and a fluid outflow tube routed through the proximal end of the handle and in fluid communication with another one of the at least one fluid lumen.

47. The device of claim 45, wherein the diameter of the working lumen is larger than the diameter of the other lumens.

48. The device of claim 44, further comprising a modular manifold integrally formed on a proximal end of the introducer tube and having a corresponding plurality of lumens aligned with the plurality of lumens in the introducer tube, the manifold removably connectable to the handle such that the manifold and introducer tube are interchangeable in the handle with other manifolds and introducer tubes.

49. The device of claim 44, wherein a separate imaging device is inserted through the handle and positioned in a predetermined one of the plurality of lumens, the imaging device having an interface with the handle such that each of the imaging device and the medical introducer is movable independent of the other.

50. The device of claim 44, wherein a separate working channel device, comprising an elongate working channel tube having at least one lumen extending the length thereof defining a working channel and a position controller for controlling the position of the working channel tube, is removably connected to the handle and positioned in another predetermined one of the plurality of lumens such that each of the medical introducer, the imaging device, and the working channel device is movable independent of the other.

51. The device of claim 44, wherein the device is disposable.

52. The device of claim 44, wherein at least a portion of the device is translucent such that passage of materials therethrough is viewable.

53. The device of claim 44, the introducer tube further comprising a proximal portion having a first durometer and a distal portion having a second durometer, wherein the second durometer is lower than the first durometer so as to allow deflection of the distal portion for controllable access to a target area in the interior body region.

54. The device of claim 53, wherein the introducer tube distal portion further comprises a distal tip having a first diameter smaller than a second diameter of the remainder of the introducer tube, the smaller first diameter adapted to seal about a device extending beyond the distal tip.

55. The device of claim 53, wherein the introducer tube further comprises a fluid lumen comprising a wall having a third durometer that is higher than the second durometer of the distal portion so as to prevent collapsing of the fluid lumen when the distal portion of the introducer tube is deflected.

56. The device of claim 44, wherein the handle further comprises a size adapted to be readily held in a hand of a user.

57. The device of claim 44, wherein the handle further comprises a plurality of raised grips on an outside surface of the handle.

58. A kit, comprising at least one of:
a medical introducer comprising a handle and an elongate introducer tube extending therefrom and having a plurality of lumens extending longitudinally therein, the medical introducer insertable into an interior body region of a patient;
a separate imaging device insertable through the handle and positionable in a predetermined one of the plurality of lumens, the imaging device having an interface with the handle such that each of the imaging device and the medical introducer is movable independent of the other; and
a separate working channel device comprising an elongate working channel tube having at least one lumen extending the length thereof defining a working channel and a position controller for controlling the position of the working channel tube, the working channel device removably connectable to the handle and positionable in another predetermined one of the plurality of lumens such that each of the medical introducer, the imaging device, and the working channel device is movable independent of the other.

59. The kit of claim 58 wherein the medical introducer handle comprises an oval-shaped ring of material having an open interior, a proximal end configured to receive at least one fluid tube and the imaging device therethrough, and a distal end adapted to connect to the introducer tube.

60. The kit of claim 58, wherein the plurality of lumens further comprises a scope lumen, at least one working lumen, and at least one fluid lumen separate from the scope lumen and the at least one working lumen.

61. The kit of claim 60, wherein the medical introducer further comprises a fluid inflow tube routed through the proximal end of the handle and in fluid communication with one of the at least one fluid lumen and a fluid outflow tube routed through the proximal end of the handle and in fluid communication with another one of the at least one fluid lumen.

62. The kit of claim 58, wherein the medical introducer further comprises a modular manifold integrally formed on a proximal end of the introducer tube and having a corresponding plurality of lumens aligned with the plurality of lumens in the introducer tube, the manifold removably connectable to the introducer handle such that the manifold and introducer tube are interchangeable in the handle with other manifolds and introducer tubes.

63. The kit of claim 62, further comprising a plurality of manifolds and introducer tubes.

64. The kit of claim 58, wherein at least the medical introducer and the working channel device are each disposable.

65. The kit of claim 58, wherein at least a portion of the medical introducer and at least a portion of the working channel device is translucent such that passage of materials therethrough is viewable.

66. The kit of claim 58, the introducer tube further comprising a proximal portion having a first durometer and a distal portion having a second durometer, wherein the second durometer is lower than the first durometer so as to allow deflection of the distal portion for controllable access to a target area in the interior body region.

67. The kit of claim 58, wherein the working channel device further comprises a steerable working channel device, and wherein the working channel tube comprises a flexible distal portion adapted for steering to selected positions.

68. The kit of claim 67,
wherein the position controller is operably connected to the working channel tube distal portion and slidable within the introducer handle for moving the working channel tube distal portion in distal and proximal directions, and
wherein the position controller is actuatable to steer the flexible distal portion of the working channel tube in predetermined directions and amounts.

69. The kit of claim 58, wherein the imaging device further comprises an endoscopic cannula, a light delivery mechanism, and a imaging system.

70. The kit of claim 58, wherein the light delivery mechanism further comprises light emitting diodes, light delivery fibers, or light emitting diodes and light delivery fibers.

71. The kit of claim 58, further comprising an accessory device support removably connectable to an outside surface of a scope connector on a proximal end of the introducer handle and comprising a carrier arm for supporting an upper part of a body of a separate medical device to be used with the medical introducer and a slide member for slidably supporting a lower part of the body of the separate medical device for stabilizing placement of the separate medical device in the interior body region.

72. The kit of claim 58 comprising the medical introducer and the working channel device.

73. The kit of claim 72 wherein the medical introducer comprises the working channel device inserted therein.

74. A method, comprising:
inserting into an interior body region of a patient a medical introducer comprising a handle and an elongate introducer tube extending therefrom and having a plurality of lumens extending longitudinally therein;
inserting a separate imaging device through the handle and positioning the imaging device in a predetermined one of the plurality of lumens;
positioning the imaging device in a selected position within the interior body region;
producing an image from within the interior body region;
removably connecting to the handle a separate working channel device comprising an elongate working channel tube having at least one lumen extending the length thereof defining a working channel and a position controller for controlling the position of the working channel tube, and positioning the working channel in another predetermined one of the plurality of lumens; and
moving one of the group of the medical introducer, the imaging device, and the working channel device independent of the others of the group.

75. The method of claim 74, the medical introducer handle further comprising an oval-shaped ring of material having an open interior, the method further comprising connecting a distal end of the handle to the introducer tube.

76. The method of claim 74, the medical introducer further comprising a modular manifold integrally formed on a proximal end of the introducer tube and having a corresponding plurality of lumens aligned with the plurality of lumens in the introducer tube, the method further comprising removably connectable the manifold to the introducer handle.

77. The method of claim 76, further comprising interchanging the manifold and introducer tube in the handle with other manifolds and introducer tubes.

78. The method of claim 74, further comprising supplying fluid to the interior body region through a fluid inflow tube and a first dedicated fluid lumen in the introducer tube and removing fluid from the interior body region through a second dedicated fluid lumen in the introducer tube and a fluid outflow tube.

79. The method of claim 74, wherein at least a portion of the medical introducer and at least a portion of the working channel device is translucent, the method further comprising viewing passage of materials through the medical introducer and the working channel device.

80. The method of claim 74, the introducer tube further comprising a proximal portion having a first durometer and a distal portion having a second durometer lower than the first durometer, the method further comprising deflecting the distal portion of the introducer tube for controlling access to a target area in the interior body region.

81. The method of claim 80, further comprising sealing a distal tip of the introducer tube about a device extending beyond the distal tip.

82. The method of claim 74, the working channel device further comprising a steerable working channel device, and the working channel tube further comprising a flexible distal portion, the method further comprising controlling the steering of the flexible distal portion of the working channel device in predetermined directions and amounts to selected positions in the interior body region independent of movement of the medical introducer and the imaging device.

83. The method of claim 82, wherein, the position controller is operably connected to the working channel tube distal portion, the method further comprising sliding the position controller within the introducer handle for moving the working channel tube distal portion in distal and proximal directions.

84. The method of claim 82, wherein the position controller further comprises a soft material on interfacing surfaces of rotatable portions of the position controller, the method further comprising automatically braking rotation of the rotatable portions so as to maintain a position of the working channel distal portion when the position controller is released by a user.

85. The method of claim 74, the working channel tube further comprising a proximal portion having a first durometer and a distal portion having a second durometer lower than the first durometer, the method further comprising deflecting the distal portion of the working channel tube for controlling access to a target area in the interior body region.

86. The method of claim 85, further comprising sealing a distal tip of the working channel tube about a device extending beyond the distal tip.

87. The method of claim 74, further comprising:
securely connecting the imaging device to a scope connector located on an opposite side of the handle from the introducer tube and longitudinally aligned with the one of the plurality of lumens in the introducer tube; and
rotating the imaging device independent of movement of the medical introducer.

88. The method of claim 74, wherein the imaging device further comprises a proximal portion having a first durometer and a distal portion having a second durometer lower than the first durometer, the method further comprising deflecting the distal portion of the introducer tube for controlling access to a target area in the interior body region.

89. The method of claim 74, further comprising delivering light to the interior body region with light emitting diodes, light delivery fibers, or light emitting diodes and light delivery fibers.

90. The method of claim 74, further comprising placing a separate medical device in the interior body region by using an accessory device support removably connected to the introducer handle and comprising a carrier arm for supporting an upper part of a body of the separate medical device and a slide of the body of the separate medical device.

91. The method of claim 74, further comprising performing a medical procedure in the interior body region through the working channel device.

92. The method of claim 91, wherein the performing a medical procedure further comprises performing a gynecological procedure.

93. The method of claim 91, wherein the performing a medical procedure further comprises performing a spinal procedure.

94. The method of claim 74, further comprising disposing of the medical introducer and the working channel device.
